(19) United States
(12) Patent Application Publication
(10) Pub. No.: US 2008/0234236 A1
(43) Pub. Date: Sep. 25, 2008

(54) REDUCTION OF ADVERSE EVENTS AFTER PERCUTANEOUS INTERVENTION BY USE OF A THROMBIN RECEPTOR ANTAGONIST

(76) Inventors: Enrico P. Veltri, Princeton, NJ (US); John T. Strony, Lebanon, NJ (US); Madhu Chintala, Colts Neck, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 12/051,504

(22) Filed: Mar. 19, 2008

Related U.S. Application Data
(60) Provisional application No. 60/896,738, filed on Mar. 23, 2007.
(60) Provisional application No. 60/932,628, filed on May 31, 2007.
(60) Provisional application No. 60/985,051, filed on Nov. 2, 2007.

(51) Int. Cl. A61K 31/443; A61K 31/5377; A61K 31/4365; A61K 31/60

(52) U.S. Cl. 514/165; 514/337; 514/232.8; 514/301

(57) ABSTRACT

Disclosed are methods of preventing adverse clinical events in a patient undergoing a percutaneous coronary intervention procedure or a peripheral percutaneous interventional procedure comprising administering a therapeutically effective amount of a thrombin receptor antagonist, such as SCH 530348, to the patient. Administration of a loading dose of about 40 mg of SCH 530348 in as little as one hour prior to the procedure can result in therapeutically effective levels of platelet aggregation.

### CROSS-REFERENCE TO RELATED APPLICATIONS

The present application claims the benefit of U.S. Provisional Application No. 60/896,738 filed Mar. 23, 2007, U.S. Provisional Application No. 60/1932,628 filed May 31, 2007, and U.S. Provisional Application No. 60/985,051 filed Nov. 2, 2007, all of which are herein incorporated in their entirety.

### BACKGROUND OF THE INVENTION

Despite aggressive antiplatelet and antithrombotic therapies, periprocedural adverse clinical events continue to occur among patients undergoing percutaneous coronary intervention (“PCI”), e.g., coronary angioplasty, stent implantation and atherectomy. The most serious adverse clinical results associated with PCI are death, myocardial infarction (“MI”) and aortic dissection. The discovery of agents that reduce clinical events without adding a bleeding liability has been elusive to date.

A variety of drugs that interfere with platelet function decrease morbidity and mortality associated with thrombotic events (e.g., MI, stroke, vascular death or the need for revascularization) in people with vascular disease. Despite current antiplatelet therapies, patients remain at risk for these serious thrombotic events.

Schering-Plough Corporation is currently developing a thrombin receptor antagonist (“TRA”) (SCH 530348) for, inter alia, the treatment of acute coronary syndrome (“ACS”). SCH 530348 targets a novel mechanism of inhibition of platelet aggregation; that is, SCH 530348 inhibits platelet aggregation by selectively binding to a G-coupled protease-activated receptor, PAR-1, the primary thrombin receptor on human platelets. The serine protease, thrombin, is the most potent activator of platelets. Therefore, an agent that selectively interferes with the cellular action of thrombin at PAR-1 may be useful in the treatment, or prevention, of arterial thrombotic disease. Moreover, a thrombin receptor antagonist will not interfere with the fibrin-generating actions of thrombin or collagen-induced platelet aggregation. As a result, a thrombin receptor antagonist may have the benefit of efficacy without incrementally increasing bleeding.

Chemically, SCH 530348 is: ethyl [(1R,3aR,4aR,6R,8aR,9S,9aS)-9-[(E)-2-[5-(3-fluorophenyl)-2-pyridinyl]ethenyl]-dodecahydro-1-methyl-3-oxonaphtho[2,3-c]furan-6-yl] carbamate bisulfate, and has the following structural formula:

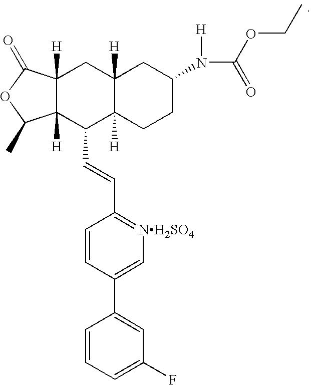

SCH 530348 is disclosed in U.S. Pat. No. 7,304,078, crystalline forms of the bisulfate salt are disclosed in U.S. Pat. No. 7,235,567, formulations of SCH 530348 are disclosed in U.S. application Ser. Nos. 11/771,520; 11/771,571; 11/860,165; and 11/960,320 and methods of treating a variety conditions are disclosed in U.S. application Ser. Nos. 10/705,282; 11/613,450; 11/642,505; and 11/642,487, all of which are herein incorporated in their entirety. The use of thrombin receptor antagonists in preventing adverse cardiovascular events related to cardiopulmonary bypass procedures is taught in U.S. application Ser. No. 11/613,450, the entirety of which is herein incorporated.

### BRIEF SUMMARY OF THE INVENTION

It is an object of the invention to provide a method of preventing an adverse clinical event in a patient who is to undergo a percutaneous coronary intervention procedure comprising administering a therapeutically effective amount of a thrombin receptor antagonist to the patient.

In some embodiments, said adverse clinical event is a myocardial infarction, urgent revascularization, or ischemia requiring hospitalization.

In some embodiments, said thrombin receptor antagonist is SCH 530348.

In some embodiments, said therapeutically effective amount is administered as a loading dose of about 1 mg to about 5 mg.

In some embodiments, said therapeutically effective amount is administered as a loading dose of about 2.5 mg.

In some embodiments, the method further comprises administering a maintenance dose of SCH 530348 to the patient once a day.

In some embodiments, said maintenance dose is about 20 mg to about 40 mg.

In some embodiments, said maintenance dose is about 40 mg.

In some embodiments, said thrombin receptor antagonist is the bisulfate salt of SCH 530348.

In some embodiments, said thrombin receptor antagonist is selected from the group consisting of

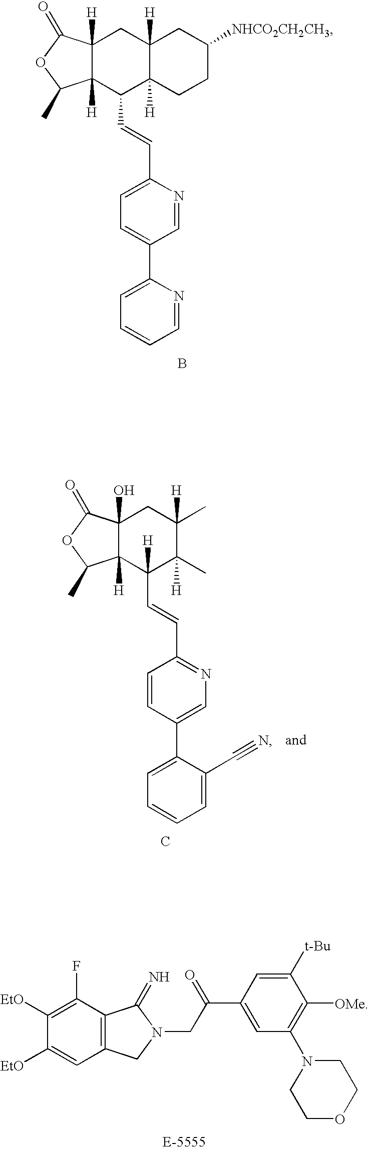

In some embodiments, the method further comprises administering to said patient an effective amount of a non-steroidal anti-inflammatory.

In some embodiments, said non-steroidal anti-inflammatory is aspirin.

In some embodiments, the method further comprises administering to said patient an effective amount of an ADP antagonist.

In some embodiments, said ADP antagonist is clopidogrel.

In some embodiments, said ADP antagonist is prasugrel.

In some embodiments, said thrombin receptor antagonist does not cause significant bleeding.

In some embodiments, said bleeding is a TIMI major/minor bleed, a TIMI major bleed, or a TIMI minor bleed, or a combination thereof.

In some embodiments, said percutaneous coronary intervention procedure is selected from the group consisting of balloon angioplasty, implantation of a stent, atherectomy, and brachytherapy.

In some embodiments, said administration has no substantial effect on ADP-induced platelet aggregation.

In some embodiments, said administration has no substantial effect on AA-induced platelet aggregation.

In some embodiments, said administration has no substantial effect on collagen-induced platelet aggregation.

It is another object of the invention to provide a method of preventing an adverse clinical event in a patient who is to undergo a percutaneous interventional procedure to treat peripheral artery disease comprising administering a therapeutically effective amount of a thrombin receptor antagonist to the patient.

In some embodiments, said thrombin receptor antagonist is SCH 530348.

In some embodiments, said therapeutically effective amount is administered as a loading dose of about 1 mg to about 5 mg.

In some embodiments, said therapeutically effective amount is administered as a loading dose of about 2.5 mg.

In some embodiments, the method further comprises administering a maintenance dose of SCH 530348 to the patient once a day.

In some embodiments, said maintenance dose is about 20 mg to about 40 mg.

In some embodiments, said maintenance dose is about 40 mg. in some embodiments, said thrombin receptor antagonist is the bisulfate salt of SCH 530348.

In some embodiments, said thrombin receptor antagonist is selected from the group consisting of

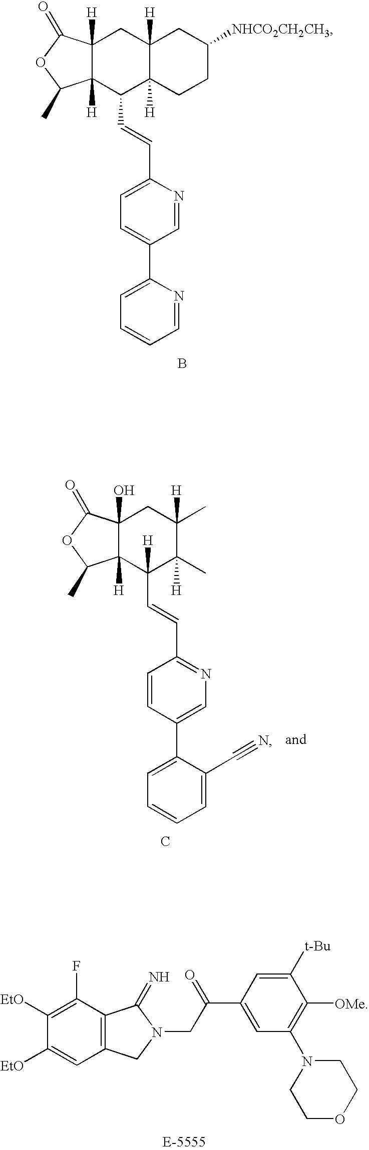

In some embodiments, said method further comprises administering to the patient an effective amount of a non-steroidal anti-inflammatory.

In some embodiments, said non-steroidal anti-inflammatory is aspirin.

In some embodiments, the method further comprises administering to the patient an effective amount of an ADP antagonist.

In some embodiments, said ADP antagonist is clopidogrel, In some embodiments, said ADP antagonist is prasugrel.

In some embodiments, said percutaneous interventional procedure is selected from the group consisting of angioplasty, plaque excision and bypass grafting.

It is yet another object of the invention to provide a method of achieving at least 80% platelet inhibition in a patient who is to undergo a percutaneous coronary interventional procedure or a peripheral percutaneous interventional procedure comprising administering to the patient a loading dose of about 40 mg of SCH 530348 at least one hour prior to the start of the procedure.

These and other objectives will be elucidated in the following description.

### BRIEF DESCRIPTION OF THE FIGURES

FIG. 1 is a flow chart showing the Phase 2 study design.

FIG. 2 is a histogram of the percent of subjects in the PCI cohort displaying TIMI major/minor bleeding.

FIG. 3 is a histogram of the percent of subjects in the PCI cohort displaying TIMI bleeding.

FIG. 4 is a histogram of the percent of subjects in the PCI cohort displaying 60-day death or MACE.

FIG. 5 is histogram of the percent of subjects in the PCI cohort displaying 60-day death or MI.

FIG. 6 shows the incidence of MI in patients in the PCI cohort over a 7-day period.

FIG. 7 is a histogram of the percent of subjects with at least 80% inhibition of platelet aggregation using TRAP based on three loading doses, grouped by dose.

FIG. 8 is a histogram of the percent of subjects with at least 80% inhibition of platelet aggregation using TRAP based on three loading doses, grouped by time.

FIG. 9 is a histogram of the percent of subjects with at least 80% inhibition of platelet aggregation using TRAP based on three maintenance doses, grouped by dose.

FIG. 10 is a histogram of the percent of subjects with at least 80% inhibition of platelet aggregation using TRAP based on three maintenance doses, grouped by time.

FIG. 11 is a histogram of the percent of subjects with at least 80% inhibition of platelet aggregation using TRAP based on three loading doses, grouped by dose.

FIG. 12 is a histogram of TRAP-induced platelet aggregation over time for each of 3 loading doses, grouped by time.

FIG. 13 is a histogram of TRAP-induced platelet aggregation over time for each of 3 maintenance doses, grouped by time.

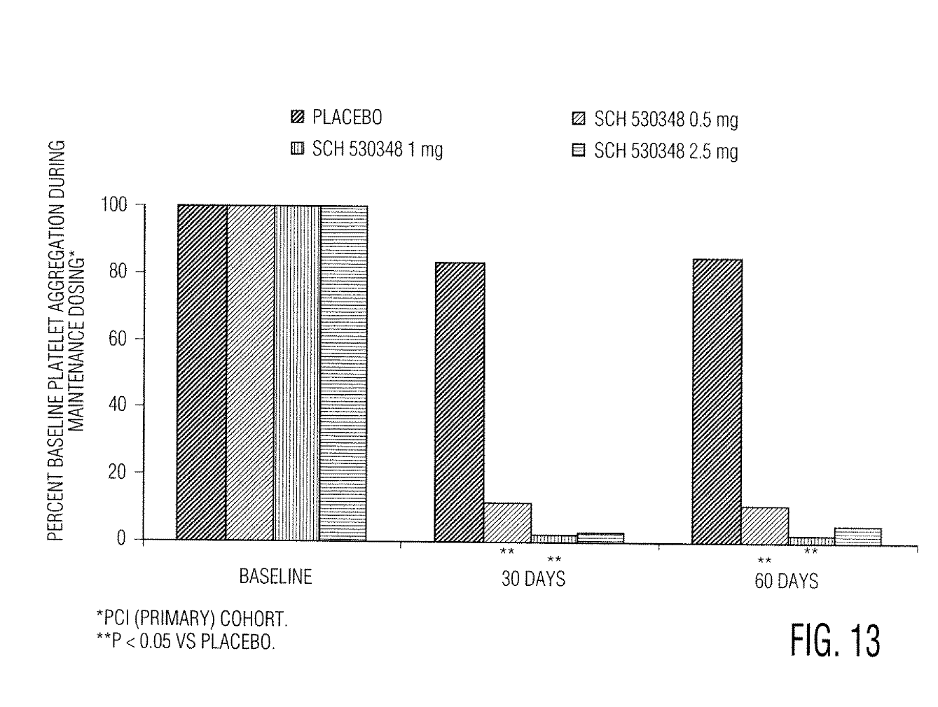

FIG. 14 is a histogram of ADP-induced platelet aggregation over time for each of 3 loading doses.

FIG. 15 is a histogram of ADP-induced platelet aggregation over time for each of 3 maintenance doses.

FIG. 16 is a histogram of AA-induced platelet aggregation over time for each of 3 loading doses.

FIG. 17 is a histogram of AA-induced platelet aggregation over time for each of 3 maintenance doses.

FIG. 18 is a histogram of collagen-induced platelet aggregation over time for each of 3 maintenance doses at a collagen concentration of 5 μg/ml.

FIG. 19 is a histogram of collagen-induced platelet aggregation over time for each of 3 loading doses at a collagen concentration of 5 μg/ml.

FIG. 20 is a histogram of collagen-induced platelet aggregation over time for each of 3 loading doses at a collagen concentration of 50 μg/ml.

FIG. 21 is a histogram of collagen-induced platelet aggregation over time for each of 3 maintenance doses at a collagen concentration of 50 μg/ml.

FIG. 22 is a comparison of the pharmacokinetic curve of blood level concentration of SCH 530348 and percent of platelet aggregation over time following administration of each of 3 loading doses.

### DETAILED DESCRIPTION OF THE INVENTION

As an effective antiplatelet therapy, thrombin receptor antagonists may have utility in the prevention of adverse clinical events associated with PCI. PCI procedures (or percutaneous coronary interventional procedures) include balloon angioplasty, implantation of stents (bare metal or drug-coated), rotational or laser atherectomy (a process in which a blood clot/plaque is removed from inside the vessel), and brachytherapy (treatment with radiation to inhibit restenosis).

Schering-Plough Corp. is developing SCH 530348, which is a selective inhibitor of the primary thrombin receptor, PAR-1 (protease-activated receptor-1), on human platelets. Consistent with its inhibitory effects on the thrombin receptor, SCH 530348 inhibits TRAP (thrombin receptor activating peptide) stimulated human platelet aggregation (IC₅₀=15 nM). Current clinical development of SCH 530348 is directed toward approval of a therapy that is adjunctive to current standard of care, e.g., the ADP antagonist clopidogrel and aspirin. However, an avoidance of bleeding risk by administration of an appropriate thrombin receptor antagonist as a monotherapy may have potential advantages over some of the currently approved antiplatelet drugs such as Plavix® (clopidogrel) and Ticlid® (ticolpidine) in certain situations such as PCI. Furthermore, the standard of care may evolve to encompass other ADP antagonists such as prasugrel. The treatment of PCI patients by administration of a thrombin receptor antagonist as adjunctive therapy to the standard of care encompasses future standards of care such as prasugrel, or any other ADP antagonist, and aspirin.

Acute coronary syndrome (“ACS”) is an umbrella term used to cover any group of clinical symptoms compatible with acute myocardial ischemia, including unstable angina, and non-ST segment elevation myocardial infarction (MI) and ST segment elevation MI. Acute myocardial ischemia is associated with chest pain due to insufficient blood supply to the heart muscle that results from coronary artery disease (also called coronary heart disease). Secondary prevention entails the treatment of patients who have had a heart attack or stroke to prevent another cardiovascular or cerebrovascular event. Peripheral arterial disease (“PAD”), also known as peripheral vascular disease (“PVD”), is a very common condition affecting 12-20 percent of Americans age 65 and older, PAD develops most commonly as a result of atherosclerosis, which occurs when cholesterol and scar tissue build up, forming plaque inside the arteries that narrows and clogs the arteries. The clogged arteries cause decreased blood flow to the legs, which can result in pain when walking, and eventually gangrene and amputation. The avoidance of an incremental bleeding liability introduced by the treatment of these cardiovascular conditions would be highly advantageous to affected patients, as these patients may be at a lessened ability to tolerate excessive bleeding given that their cardiovascular systems may already be stressed, and they may be of advanced age.

In addition to blocking PAR-1 receptors on platelets, SCH 530348 also inhibits PAR-1 receptors on other cells, including the endothelial cells, smooth muscle cells, neutrophils, leukocytes and monocytes. In published animal studies using either PAR-1 antagonists or PAR-1 KO mice, several investigators have demonstrated anti-inflammatory effects, including in inflammatory bowel diseases. Thus, thrombin receptor antagonists may have an anti-inflammatory action that could be beneficial to PCI patients who may suffer an inflammatory response from the angioplasty procedure. CD40 ligand and C-reactive proteins are considered biomarkers for inflammation, and will be evaluated in Phase 3 studies.

The results of certain clinical trials regarding SCH 530348 are described below.

### Monkey Bleeding Liability Studies

Any novel antiplatelet agent should optimally avoid incremental bleeding risk relative to the current standard of care. The bleeding risk of SCH 530348 was evaluated in anesthetized cynomoigus monkeys in which SCH 530348 was administered alone and along with aspirin and clopidogrel. Bleeding assessment was conducted following administration of single doses of SCH 530348 (1 mg/kg) and/or aspirin (10 mg/kg, ASA) and clopidogrel (2 mg/kg). Four groups of monkeys were dosed orally with either vehicle (the medium used to dissolve the drug, 0.4% methylcellulose) or drug (n=5-6/group): Group I: vehicle, Group II: SCH 530348 (1 mg/kg), Group III ASA (10 mg/kg) plus clopidogrel (2 mg/kg) and Group IV: combination of SCH 530348, ASA plus clopidogrel. Doses of various drugs used inhibited their respective platelet pathways by >90% as assessed by ex-vivo platelet aggregation in whole blood. Two hours after dosing, animals were anesthetized and bleeding risk was assessed by forearm template bleeding time and surgical blood loss from a femoral incision site.

Template bleeding time was assessed as follows. The forearm of the monkey was shaved of hair and a precision cut (5 mm long and 1 mm deep) was made on the skin with a Simplate® bleeding device (Organon Tehnika), The duration of bleeding by blotting blood onto a filter paper at the incision site was determined. When blood was no longer absorbed onto the filter paper, bleeding was considered “stopped.” The time from initiation of the cut to when bleeding stopped is defined as the template bleeding time. In these studies bleeding time was assessed on two occasions at approximately 3.5 and 4 hrs after oral dosing with the drug.

Surgical blood loss was assessed as follows. The femoral artery and femoral vein on the hind leg of the anesthetized monkey were surgically isolated by cutting with a scalpel blade. In addition, two 0.5 cm cuts were made on the sartorius muscle in the femoral area with the Simplate® bleeding device (Organon Teknika) to initiate bleeding. To determine the surgical blood loss, a gauze pad was placed in the femoral surgical site and blood lost at this site was adsorbed onto the gauze. The gauze was replaced with a fresh one at 30 min. Two 30- min interval collections were carried out. The gauze containing the blood was immersed into 5 ml of Drabkin's reagent (Sigma Chemical Co.) which lyses the red blood cells and forms a colored reaction product with the hemoglobin in the red blood cells. A small sample was read in a spectrophotometer (at 550 nM) and the volume of blood loss was derived from a standard curve. The standard curve was established with known amounts of blood collected at the end of the experiment for each animal.

The results of these monkey bleeding studies are summarized in Table A.

TABLE A

| | Group I | Group II | Group III | Group IV |
|---|---|---|---|---|
| Template | 3.4 ± 0.32 | 4.9 ± 0.99 | 23.2 ± 3.98* | 21.86 ± 4.3* |
| bleeding | | | | |
| time (min) | | | | |
| Surgical | 0.13 ± 0.05 | 0.18 ± 0.03 | 2.00 ± 0.28* | 2.03 ± 0.23* |
| blood loss | | | | |
| (ml/hr) | | | | |

*<0.05 vs. Vehicle group.
Data are presented as Mean ± standard error margin (“SEM”).

Based on the data in Table A, administration of vehicle or SCH 530348 resulted in a mean template bleeding time of 3.4 and 4.9 minutes and surgical blood loss of 0.13 and 0.18 ml/hr in Groups I and II, respectively. The bleeding times and blood loss observed in Groups I and II are not statistically different from each other, demonstrating that SCH 530348 at a dose of 1 mg/kg does not have any bleeding risk over that of the vehicle. Administration of aspirin plus clopidogrel in Group III resulted in a marked increase in template bleeding time of 23.2 min vs. 3.4 (Group I) and an increase in surgical blood loss of 2.00 vs. 0.13 ml/hr (Group I). Both of these increases were statistically significant. Thus, a bleeding risk was demonstrated for aspirin plus clopidogrel in Group III. However, co-administration of SCH 530348 with aspirin and clopidogrel in Group IV did not result in any additional incremental bleeding risk as assessed by template bleeding times (23.2 vs. 21.86 min in Groups III and IV, respectively) or surgical blood loss (2.00 vs. 2.03 ml/hr in Groups III and IV, respectively) over that observed in the aspirin plus clopidogrel group (Group III).

These results demonstrate that there was no bleeding risk associated with administration of SCH 530348 alone. Furthermore, SCH 530348 did not exacerbate the prolonged bleeding associated with ASA and clopidogrel. These data support the proposition that SCH 530348 can be added to the current standard of care for the treatment of atherothrombosis without incurring an added bleeding risk.

The conclusion drawn from these data is that administration of SCH 530348 at a dose of 1 mg/kg did not cause any bleeding risk in monkeys. Based on an average weight of 70 kg for adults, this dose would equate to a human dose of approximately 70 mg. Thus, in humans SCH 530348 at doses up to 70 mg should not have any bleeding risk when administered alone or when co-administered with aspirin and clopidogrel. In sum, results of these pharmacology studies demonstrate that SCH 530348:
Inhibits TRAP-driven platelet aggregation by 100% after an oral dose of 0.1 mg/kg in cynomolgus monkeys (ex-vivo); and,
Has no evidence of bleeding liability in cynomolgus monkeys when administered alone or with aspirin/clopidogrel.

SCH 530348 has activity in the hERG voltage clamp assay (IC₅₀˜341 nM). However, no evidence of QT prolongation occurred based upon either action potential duration in dog Purkinje fibers (in vitro) or in the monkey safety pharmacology study.

In a monkey surgical bleeding liability study, there was no prolongation of bleeding observed with SCH 530348 administered alone or when administered with aspirin and clopidogrel. Bleeding assessment was conducted following administration of single doses of SCH 530348 (1 mg/kg) and/or aspirin (10 mg/kg) and clopidogrel (2 mg/kg).

At the doses and treatment duration studied to date, there have been no significant treatment-related changes or abnormalities in laboratory safety tests or ECGs, including no evidence of QT prolongation. Overall, the study drug was generally well-tolerated.

### Phase 2 Clinical Studies

The behavior of SCH 530348 in humans was further studied in a Phase 2 clinical development program. A key safety issue for SCH 530348 is the potential for incremental bleeding when added to standard of care with other oral antiplatelet therapies as well as parenteral antithrombotics. Therefore, a single Phase 2 study (P03573) was completed to evaluate the safety of SCH 530348 in patients at high risk of bleeding events, i.e., those patients undergoing non-emergent PCI (percutaneous coronary intervention). The goals of this Phase 2 study were to:
Evaluate the safety of SCH 530348 in addition to standard of care with respect to TIMI major and minor bleeding; and,
Observe the effects of SCH 530348 on major adverse cardiac events (“MACE”) in patients successfully completing an interventional procedure.

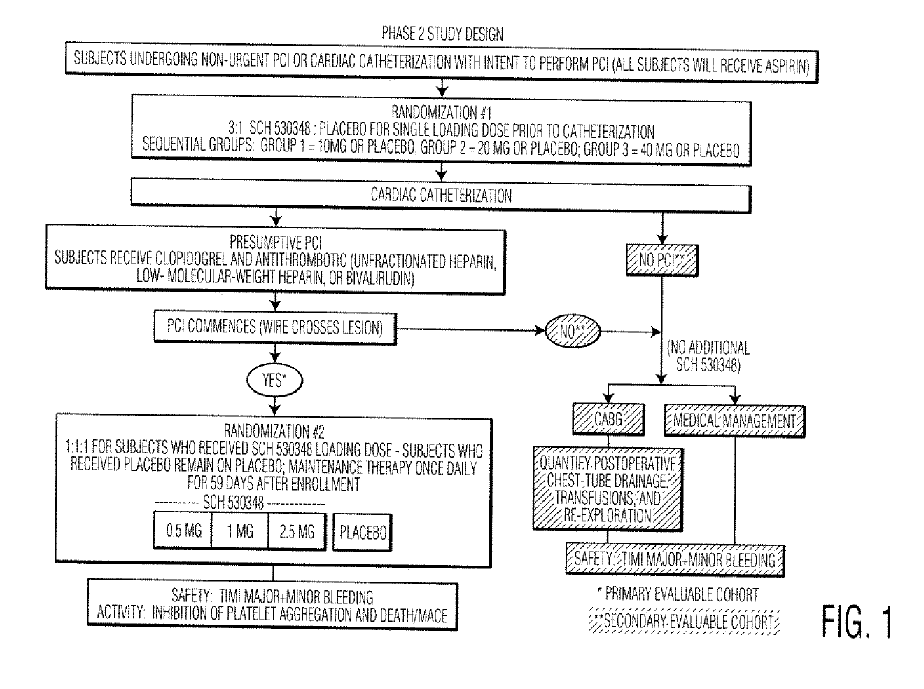

The study design is outlined in FIG. 1.

The term “loading dose” will be understood to mean a pharmaceutical composition comprising a set quantity of thrombin receptor antagonist (e.g., 10-40 mg) intended for a one-time administration. The term “maintenance dose” will be understood to mean a pharmaceutical composition comprising a lower quantity of thrombin receptor antagonist (e.g., 0.5-5 mg) intended for periodic administration (e.g., once a day) after the loading dose has been administered.

The Phase 2 study was a randomized, double-blind, placebo-controlled, multicenter, dose-escalation study in men and women with symptoms of coronary heart disease undergoing nonurgent PCI. Three loading doses (10, 20 and 40 mg) of SCH 530348 were studied (3:1 randomization of drug:placebo). Once safety and pharmacodynamics were established at a particular loading dose, another group of subjects was randomized at the next loading dose.

Following PCI, patients receiving a loading dose of SCH 530348 were randomized (1:1:1) to one of three maintenance doses of SCH 530348 (0.5, 1.0 and 2.5 mg) and received this maintenance dose for fifty-nine days post-procedure (sixty days total treatment). Patients who received a placebo loading dose received placebo for maintenance for fifty-nine days post-procedure. This group is the “primary evaluable cohort.” Those patients (˜50%) who did not undergo PCI but who received the loading dose of SCH 530348 prior to catheterization are the “secondary evaluable cohort”. Examples of some of the maintenance dose formulations dosed in the Phase 2 studies are shown in Table 1.

TABLE 1
Composition of SCH 530348 bisulfate tablets 0.5 mg, 1 mg, 2.5 mg and 10 mg

| | | Theoretical mg/tablet | | | |
|---|---|---|---|---|---|
| | | | | 2.5 mg | |
| | | 0.5 mg Tablet | 1 mg Tablet | Tablet | 10 mg Tablet |
| | | Formula TSO | Formula TSO | Formula TSO | Formula TSO |
| | | 3986 | 3943 | 3944 | 4000 |
| Ingredient | Function | FM 3986-1-1 | FM 3943-1-1 | FM 3944-1-1 | FM 3945-2-1 |
| SCH 530348 bisulfate | Active | 0.5 | 1 | 2.5 | 10 |
| Lactose Monohydrate | Diluent | 70 | 69.5 | 68 | 272 |
| (Impalpable Powder) NF | | | | | |
| Microcrystalline Cellulose | Diluent | 20 | 20 | 20 | 80 |
| NF | | | | | |
| Croscammellose Sodium | Disintegrant | 6 | 6 | 6 | 24 |
| NF | | | | | |
| Povidone K-30 USP | Binder | 3 | 3 | 3 | 12 |
| Magnesium Stearate NF | Lubricant | 0.5 | 0.5 | 0.5 | 2 |
| Non-Bovine | | | | | |
| Purified Water USP | Solvent | (—)^{a} | (—)^{a} | (—)^{a} | (—)^{a} |
| Theoretical Total Core | | 100.0 | 100.0 | 100.0 | 400.0 |
| Tablet Weight | | | | | |
| Opadry II Blue Y-30-10705 | Coating Agent | 3 | 3 | 3 | 12 |
| Purified Water USP | Solvent | (—)^{b} | (—)^{b} | (—)^{b} | (—)^{b} |
| Theoretical Total Coated | | 103.0 | 103.0 | 103.0 | 412.0 |
| Tablet Weight | | | | | |

^{a}Evaporates during the drying and coating processes
^{b}Evaporates during the coating process

The primary endpoint (directed to evaluating safety) was the combined thrombosis in myocardial infarction (“TIMI”) major and TIMI minor bleeding over sixty days in the primary evaluable cohort. Though the study was not powered to evaluate efficacy, a secondary endpoint (directed to evaluating efficacy) was the incidence of the composite of death and major adverse cardiac events (“MACE”) in the primary cohort. MACE includes myocardial infarction, urgent revascularization, and ischemia requiring hospitalization. Pharmacokinetics and pharmacodynamics were assessed at selected sites across the treatment groups. Other secondary endpoints included the incidence of TIMI major and minor bleeding in the secondary evaluable cohort i.e., those individuals who received the loading dose only.

The study size of 1600 patients was estimated based upon a 3-6% incidence of TIMI major and minor bleeding. A Safety Review Committee (SRC) reviewed the demographic and safety data of the first 923 patients enrolled into the trial. Given the lower than anticipated incidence of bleeding in the trial (1.7%), the SRC did not believe that incremental information on the safety of SCH 530348 would be generated with continued recruitment to 1600 patients and recommended ending study enrollment. A total of 1030 patients were randomized to receive either a loading dose of SCH 530348 or placebo (Table 2). Of these, 573 went on to PCI and were randomized to maintenance dosing, i.e., the primary evaluable cohort. The remaining 457 patients did not undergo PCI and thus were assigned to the secondary evaluable cohort; of these, 75 subsequently underwent coronary arterial bypass graft surgery (“CABG”).

The baseline characteristics of randomized patients were similar among the placebo and the SCH 530348 dosing groups. Most patients were men with a mean age of 64 years, and the average weight was 90 kgs. Approximately half of the patients underwent PCI and, of these, 97% (557/573) received intracoronary stent placement.

TABLE 2
Enrollment and Baseline Characteristics

| | | SCH 530348 | | | |
|---|---|---|---|---|---|
| | All Placebo | All | 10 mg | 20 mg | 40 mg |
| All Randomized | 257 | 773 | 222 | 238 | 313 |
| Primary Cohort | 151 | 422 | 129 | 120 | 173 |
| Secondary Cohort | 106 | 351 | 93 | 118 | 140 |
| Secondary Cohort | 24 | 51 | 10 | 18 | 23 |
| with CABG | | | | | |
| Male | 80% | 70% | 72% | 66% | 72% |
| Age (mean, years) | 62 | 64 | 65 | 63 | 63 |
| ≧65 years | 38% | 43% | 44% | 43% | 42% |
| Weight (mean, kg) | 91 | 90 | 89 | 92 | 90 |

Table 3 shows the distributions of antiplatelet and antithrombotic medications taken by subjects with the PCI cohort.

TABLE 3

| | | SCH 530348 | | | |
|---|---|---|---|---|---|
| | Placebo | All | 10 mg | 20 mg | 40 mg |
| | n = 151 | n = 422 | n = 129 | n = 120 | n = 173 |
| Aspirin | 148 (98%) | 416 (99%) | 127 (98%) | 117 (98%) | 172 (99%) |
| Clopidogrel | | | | | |
| All | 146 (97%) | 408 (97%) | 127 (98%) | 117 (98%) | 164 (95%) |
| 75 mg | 73 (48%) | 191 (45%) | 56 (43%) | 52 (43%) | 83 (48%) |
| 300 mg | 30 (20%) | 85 (20%) | 34 (26%) | 21 (18%) | 30 (17%) |
| 600 mg | 40 (26%) | 125 (30%) | 36 (28%) | 39 (33%) | 50 (29%) |
| Antithrombin Agent | | | | | |
| Heparin | 61 (40%) | 181 (43%) | 53 (41%) | 52 (43%) | 76 (44%) |
| Bivalrudin | 76 (50%) | 196 (46%) | 65 (50%) | 51 (43%) | 80 (46%) |
| GP IIb/IIIa | 7 (5%) | 37 (9%) | 7 (5%) | 14 (12%) | 16 (9%) |

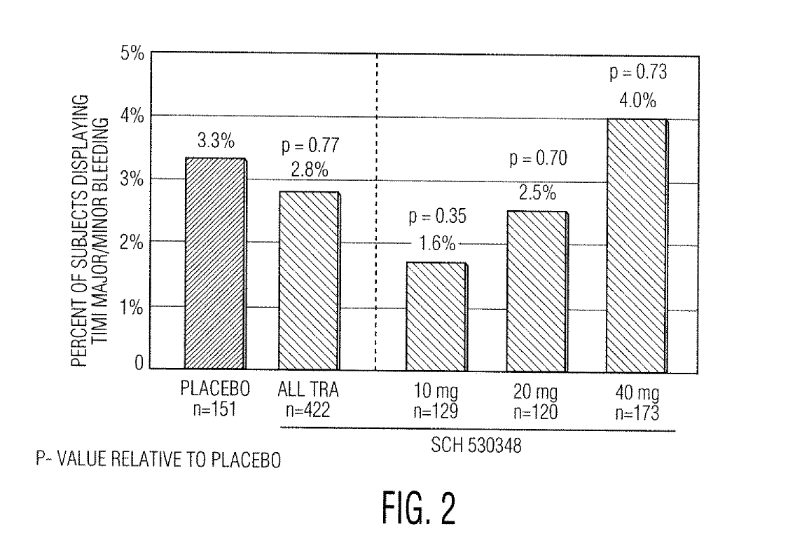

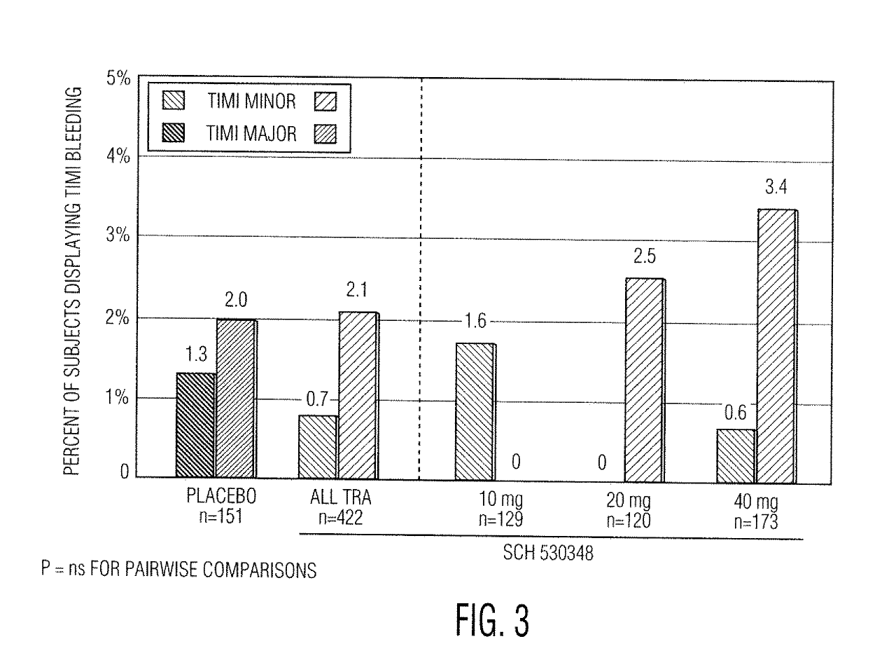

There was no statistically significant difference between SCH 530348 and placebo (2.8% vs. 3.3%) in the proportion of patients with the combined incidence of TIMI Major and Minor bleeding over 60 days in the primary cohort, i.e., the primary endpoint of the study. These data are summarized in Table 4 below. Overall, TIMI Major and Minor bleeding rates were low, and most were periprocedural, occurring during hospitalization. Additionally, when looking at the incidence of bleeding with respect to loading doses (pooled across the maintenance doses) in the primary cohort, there was no significant increase in non-TIM bleeding (41% vs. 32%) for SCH 530348 vs. placebo, respectively These data are graphically displayed in FIGS. 2 and 3.

The secondary cohort consisted of non-PCI patients who were treated medically (n=382) or underwent CABG (n=75). There were no TIMI Major bleeds, and 3 cases (<1%) of TIMI Minor bleeds were reported for the SCH 530348 treated patients in the medically treated secondary cohort. These included two vascular access site and one post-operative (surgical hip replacement) bleed.

TABLE 4
Summary of Bleeding In Primary (PCI) and Secondary
(Medically Treated) Cohorts**

| | | SCH 530348 | | | |
|---|---|---|---|---|---|
| | All Placebo | All | 10 mg | 20 mg | 40 mg |
| Primary cohort (PCI) | 151 | 422 | 129 | 120 | 173 |
| Any bleed | 53 (35%) | 177 (42%) | 48 (37%) | 52 (43%) | 77 (45%) |
| TIMI | 5 (3.3%) | 12 (2.8%) | 2 (1.6%) | 3 (2.5%) | 7 (4.0%) |
| Major/Minor* | | | | | |
| TIMI Major | 2 (1.3%) | 3 (0.7%) | 2 (1.6%) | 0 | 1 (0.6%) |
| TIMI Minor | 3 (2.0%) | 9 (2.1%) | 0 | 3 (2.5%) | 6 (3.4%) |
| non-TIMI | 48 (32%) | 170 (40%) | 46 (36%) | 51 (43%) | 73 (42%) |
| bleeding | | | | | |
| Secondary cohort | 82 | 300 | 83 | 100 | 117 |
| (medically treated) | | | | | |
| Any bleed | 4 (5%) | 31 (10%) | 7 (8%) | 12 (12%) | 12 (10%) |
| TIMI Major/Minor | 0 | 3 (1%) | 0 | 2 (2%) | 1 (1%) |
| TIMI Major | 0 | 0 | 0 | 0 | 0 |
| TIMI Minor | 0 | 3 (1%) | 0 | 2 (2%) | 1 (1%) |
| non-TIMI | 4 (5%) | 30 (10%) | 7 (8%) | 11 (11%) | 12 (10%) |
| bleeding | | | | | |

*primary safety endpoint
**excludes CABG

The use of TIMI bleeding classification alone is of limited value and can be misleading in defining bleeding risk in patients undergoing CABG because of the routine use of packed red blood cell (“PRBC”) transfusions to prime the pump and extracorporeal oxygenator, and the resultant decline in hemoglobin that results from the administration of parenteral fluids during surgery. Therefore, other clinically meaningful measures (e.g., chest tube drainage, transfusions, need for re-exploration) were examined.

There was a slight, not statistically significant, increase in the incidence of TIMI Major and Minor bleeding in SCH 530348 treated patients who underwent CABG (94% vs. 80% in placebo) (Table 5). However, there was no increase in total chest tube drainage, the proportion with >2 units PRBC transfusion, or the need for re-exploration in SCH 530348 treated patients, Overall, these findings would suggest that SCH 530348 administration is not associated with increased risk of clinically relevant bleeding during CABG.

TABLE 5

| | | SCH 530348 | | | |
|---|---|---|---|---|---|
| | Placebo | All | 10 mg | 20 mg | 40 mg |
| | n = 24 | n = 51 | n = 10 | n = 18 | n = 24 |
| Any Bleed | 24 | 52 | 10 | 18 | 24 |
| TIMI Major/ | 19 (79%) | 48 (92%) | 9 (90%) | 17 (94%) | 22 (92%) |
| Minor | | | | | |
| Non-TIMI | 8 (33%) | 18 (35%) | 3 (30%) | 6 (33%) | 9 (39%) |
| Transfusion | | | | | |
| PRBC | 11 (46%) | 32 (62%) | 8 (80%) | 9 (50%) | 15 (63%) |
| >2 Units | 5 | 9 | 2 | 2 | 5 |
| Chest Tube | | | | | |
| Drainage (ml) | 996 | 988 | 1393 | 1015 | 870 |
| Re-exploration | 3 | 2 | 1 | 0 | 1 |

The Phase 2 study was designed with the intent to capture all bleeding events irrespective of their clinical importance or severity. In both the primary and secondary evaluable cohorts, the non-TIMI bleeding events represent a broad range of origin. Details are summarized in Tables 6 and 7 below. There was a slight increase in non-TIM bleeding observed with SCH 530348 which was not statistically significant in either primary or the medically treated secondary cohorts.

TABLE 6
Summary of non-TIMI bleeding in Primary (PCI) and Secondary
(Medically treated and CABG) Cohorts

| | | SCH 530348 | | | |
|---|---|---|---|---|---|
| | All Placebo | All | 10 mg | 20 mg | 40 mg |
| Primary cohort (PCI) | 151 | 422 | 129 | 120 | 173 |
| Any bleed | 53 (35%) | 177 (42%) | 48 (37%) | 52 (43%) | 77 (45%) |
| non-TIMI | 48 (32%) | 173 (41%) | 47 (36%) | 52 (43%) | 74 (43%) |
| bleeding | | | | | |
| Secondary cohort | 82 | 300 | 83 | 100 | 117 |
| (medically treated) | | | | | |
| Any bleed | 4 (5%) | 31 (10%) | 7 (8%) | 12 (12%) | 12 (10%) |
| non-TIMI | 4 (5%) | 30 (10%) | 7 (8%) | 11 (11%) | 12 (10%) |
| bleeding | | | | | |
| Secondary cohort | 24 | 51 | 10 | 18 | 23 |
| (CABG treated) | | | | | |
| Any bleed | 24 (100%) | 51 (100%) | 10 (100%) | 18 (100%) | 23 (100%) |
| non-TIMI | 8 (33%) | 17 (33%) | 2 (20%) | 6 (33%) | 9 (39%) |
| bleeding | | | | | |

A breakdown of non-TIMI bleeding in the Primary PCI cohort is summarized in Table 7 below. There was no significant difference in the incidence of epistaxis, gingival bleeding, gastrointestinal bleeding, genitourinary bleeding, or other bleeding events that typically lead to patient non-compliance. Most non-TIMI bleeding was vascular access related or skin bruising/contusions. Importantly, treatment discontinuations were low, and no different between SCH 530348 and placebo (1% in each group).

TABLE 7

| | | SCH 530348 | | | |
|---|---|---|---|---|---|
| | Placebo | All | 10 mg | 20 mg | 40 mg |
| | n = 151 | n = 422 | n = 129 | n = 120 | n = 173 |
| Overall | 48 (32%) | 170 (40%) | 46 (36%) | 51 (43%) | 73 (42%) |
| Vascular Puncture | 20 (13%) | 64 (15%) | 16 (12%) | 22 (18%) | 26 (15%) |
| Arterial Access | 19 (13%) | 58 (14%) | 14 (11%) | 20 (17%) | 24 (14%) |
| Contusion/Bruise | 17 (11%) | 64 (15%) | 13 (10%) | 20 (17%) | 31 (18%) |
| Incidental Cuts | 10 (7%) | 39 (9%) | 5 (4%) | 16 (13%) | 18 (10%) |
| Expistaxis | 12 (8%) | 23 (5%) | 8 (6%) | 6 (5%) | 9 (5%) |
| GI | 2 (1%) | 8 (2%) | 2 (2%) | 1 (1%) | 5 (3%) |
| Gingival | 2 (1%) | 5 (1%) | 2 (2%) | 1 (1%) | 2 (1%) |
| Genitourinary | 1 (1%) | 13 (3%) | 4 (3%) | 6 (5%) | 3 (2%) |
| Discontinue for AE | 2 (1.3%) | 6 (1.4%) | 1 (0.8%) | 1 (0.8%) | 4 (2.3%) |

Overall, SCH 530348 was generally well tolerated, with 355 of 422 patients (84%) completing the sixty day treatment period compared to 135 of 151 patients (89%) on placebo. Discontinuation of study drug for any adverse event occurred in 27 patients (6%) treated with SCH 530348 and 8 patients (5%) on placebo.

Although the Phase 2 study was neither designed nor powered to assess efficacy, i.e., reduction of clinical event rates, there was a 31% relative reduction observed in the composite of death and major adverse cardiac events (MACE) at sixty days in the SCH 530348 group (5.9%) versus placebo (8.6%) in the primary cohort. This difference was not statistically significant. Other efficacy endpoints in the primary cohort are summarized in Table 8 below. All clinical events were adjudicated by a blinded Clinical Events Committee.

TABLE 8

| | | SCH 530348 | | | |
|---|---|---|---|---|---|
| | Placebo | All | 10 mg | 20 mg | 40 mg |
| | n = 151 | n = 422 | n = 129 | n = 120 | n = 173 |
| Death/MACE/Stroke | 13 (8.6%) | 26 (6.2%) | 12 (9.3%) | 6 (5.0%) | 8 (4.6%) |
| Death/MACE | 13 (8.6%) | 25 (5.9%) | 11 (8.5%) | 6 (5.0%) | 8 (4.6%) |
| Death/MI | 11 (7.3%) | 19 (4.5%) | 7 (5.4%) | 5 (4.2%) | 7 (4.0%) |
| Death | 0 | 2 (0.5%) | 1 (0.8%) | 0 | 1 (0.6%) |
| MACE | 13 (8.6%) | 24 (5.7%) | 11 (8.5%) | 6 (5.0%) | 7 (4.0%) |
| MI | 11 (7.3%) | 18 (4.3%) | 7 (5.4%) | 5 (4.2%) | 6 (3.5%) |
| Recurrent ischemia | 1 (0.7%) | 1 (0.2%) | 1 (0.8%) | 0 | 0 |
| Revascularization | 1 (0.7%) | 6 (1.4%) | 3 (2.3%) | 1 (0.8%) | 2 (1.2%) |
| Stroke | 0 | 1 (0.2%) | 1 (0.8%) | 0 | 0 |

MACE = Major Adverse Cardic Event (myocardial infarction, ischemia requiring hospitazliation, coronary revascularization)
MI = Myocardial Infarction
* = primary efficacy endpoint

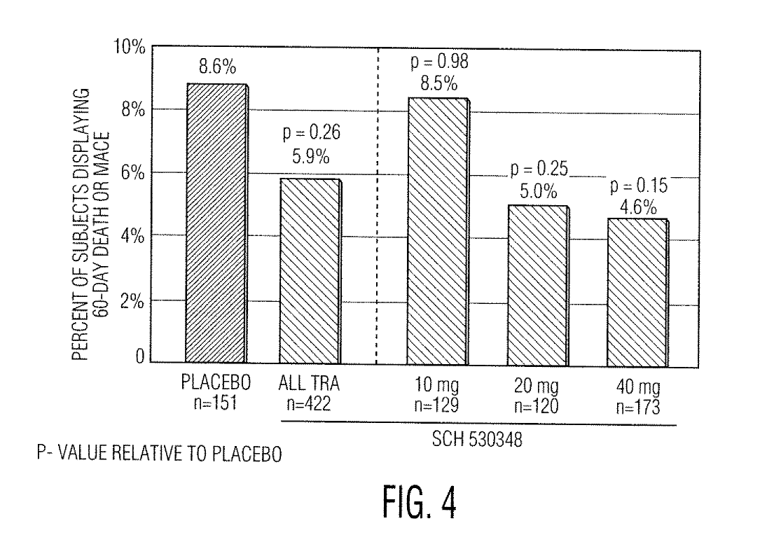

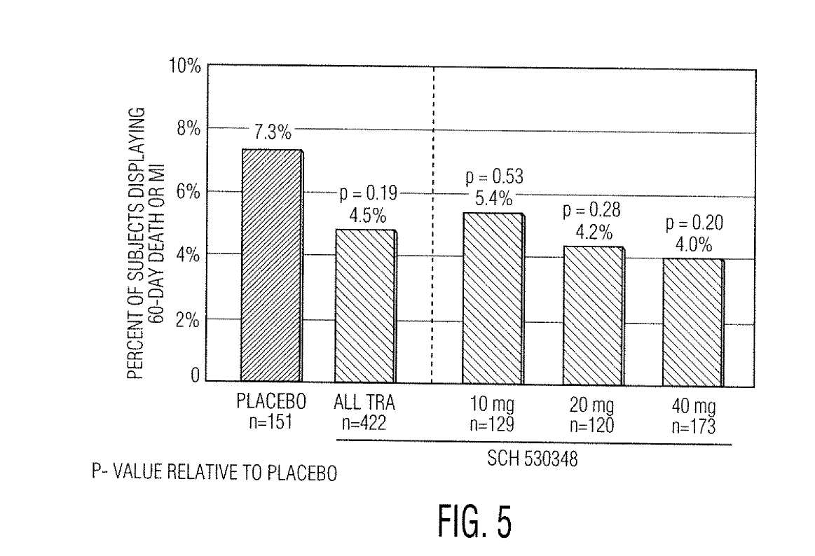

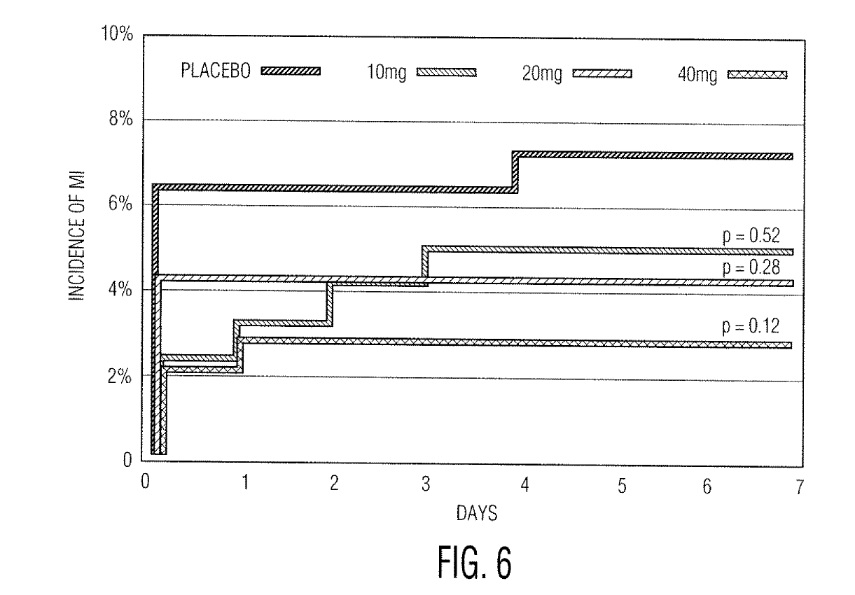

A decrease in the composite of death and MACE was observed with increasing loading doses of SCH 530348, (8.5% for 10 mg, 5% for 20 mg, and 4.6% for 40 mg), resulting in a 47% relative reduction seen with SCH 530348 40 mg versus placebo. The benefit was primarily due to the reduction in non-fatal MI events, which were predominantly periprocedural (23 of 29) non-fatal MI. The reduction in non-fatal MI also appeared dose related, (5.4% for 10 mg, 4.2% for 20 mg, 3.5% for 40 mg compared to 7.3% for placebo).These data are graphically displayed in FIGS. 4-6. Thus for the SCH 530348 40 mg loading dose cohort versus placebo, there was a 52% reduction in MI (3.5% vs. 7.3%). Most importantly, the event reduction was in the setting of ASA (aspirin) and clopidogrel background therapy. ASA was used in 98% of placebo and 99% of SCH 530348 treated groups, and 97% of all patients received clopidogrel. Thus, in the context of standard of care that includes ASA and clopidogrel administration, thrombin receptor mediated inhibition of platelet aggregation prior to PCI, a controlled setting of plaque rupture appears to translate into a decrease in periprocedural events.

Three deaths were observed in the study: two in the primary PCI cohort and one in the secondary non-PCI cohort. All were in the SCH 530348 group. One of the primary cohort deaths was due to an MI on day 138 Seventy-eight days following the completion of therapy); the second death was due to pump failure immediately following PCI. The third death occurred in a patient assigned to the secondary non-PCI cohort who received loading dose only. This death was due to a pulmonary embolus seventy-two days following SCH 530348 administration. Each of these deaths was considered by the investigator unlikely to be related to study drug.

There were five nonfatal strokes reported by the investigators in the study: one stroke in the primary PCI cohort and four strokes in the secondary cohort. All were randomized to SCH 530348. The stroke in the primary PCI cohort was a lacunar infarction that occurred forty-four days into therapy. Of the remaining four strokes, two occurred in the CABG treated and two in the medically treated secondary cohort. Of the CABG associated strokes, one occurred on the day of surgery while the second was noted thirty-six days post-operatively (aortic valve replacement as well). Of the two strokes in the medically treated cohort, one was diagnosed as a subdural hematoma resulting from a fall with head trauma and a fractured zygomatic arch three days following the single loading dose administration; the second was adjudicated as a transient ischemic attack in the setting of atrial fibrillation with no residual deficit occurring twenty-five days following the single loading dose administration. All of these events were considered unlikely to be related to study drug by the investigator.

This study was designed to test the safety and tolerability of SCH 530348 across a range of loading and maintenance doses in addition to standard of care antiplatelet therapy (aspirin and clopidogrel). Within the primary PCI cohort, SCH 530348 provided no significant increase in the incidence of TIMI Major plus Minor bleeding, with a small increase in non-TIMI bleeding which was neither statistically significant nor associated with study drug discontinuation.

Within the secondary cohort undergoing CABG, there was a numerical increase in TIMI Major plus Minor bleeding. However, this difference was not statistically significant and the clinical relevance of such findings is questionable given that the more clinically important parameters such as total chest tube drainage, PRBC transfusions of >2 units, and re-explorations were either the same or less than placebo. Non-TIMI bleeding was similar between treatment groups. In aggregate, treatment with SCH 530348 was not associated with an increased risk in bleeding.

The trial was designed as a safety study with insufficient power to assess efficacy. Thus, the benefit of SCH 530348 resulting from the reduction of clinical efficacy endpoints should be interpreted with caution. Nonetheless, there was a decrease in the frequency of the composite of death and MACE, driven primarily by reductions in myocardial infarctions, and appeared possibly dose related. Most events were clustered around the time of PCI.

### Pharmacodynamic Substudy

A pharmacodynamic substudy incorporating light aggregometry was performed under stringent physiologic conditions and read by a central core laboratory. The purpose of this substudy was twofold: to document the presence of clinically relevant inhibition of platelet aggregation defined as ≧80% inhibition of TRAP mediated platelet aggregation; and, to identify the dosing regimen that affords the greatest proportion of the study population with clinically appropriate inhibition of platelet aggregation. The term “therpeutically effective amount of a thrombin receptor antagonist” will be understood to mean that quantity of the thrombin receptor antagonist sufficient to achieve at least 80% inhibition of TRAP mediated platelet aggregation.

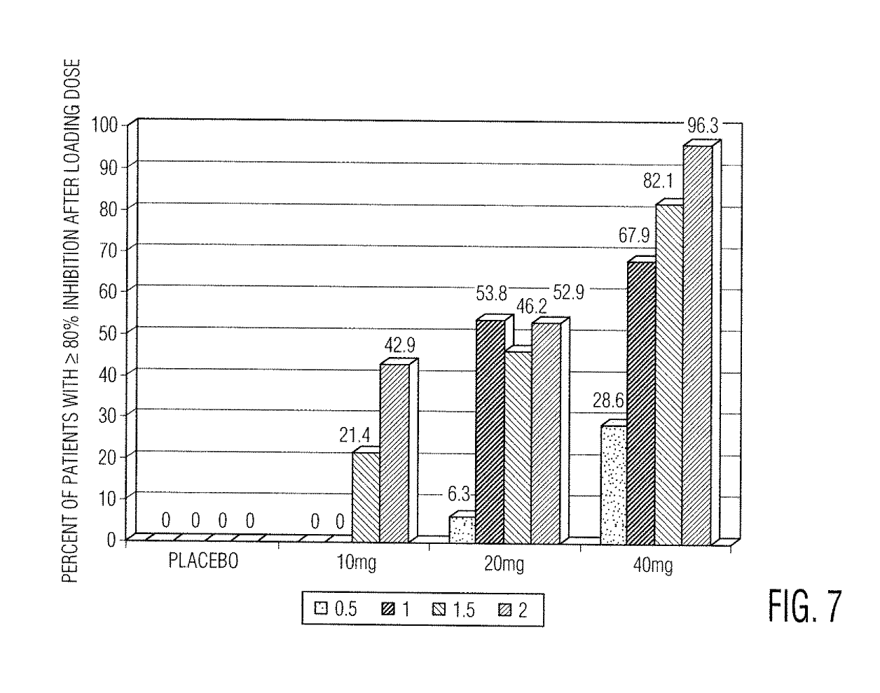

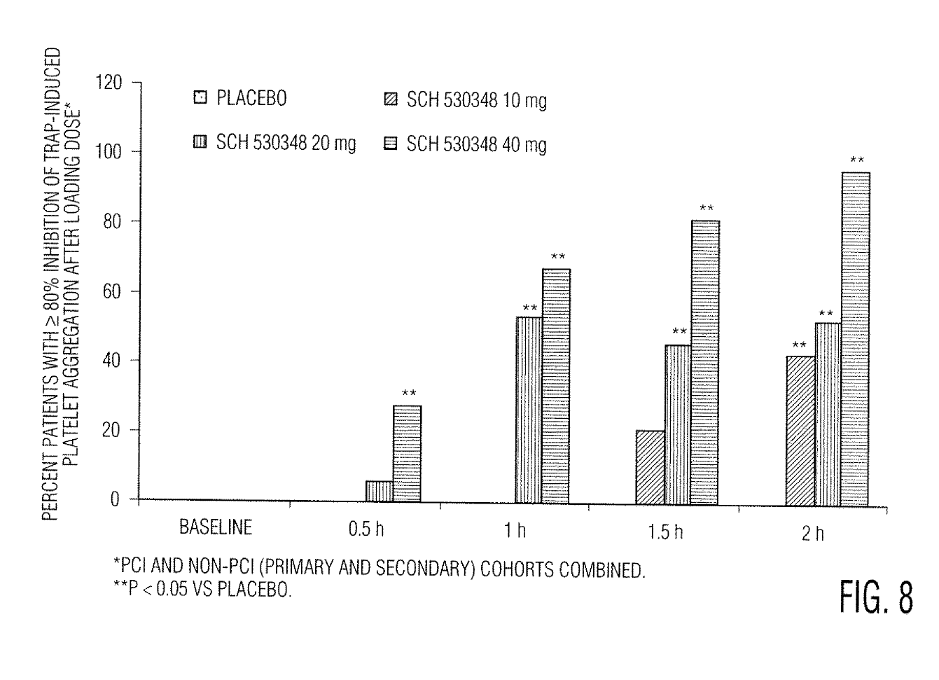

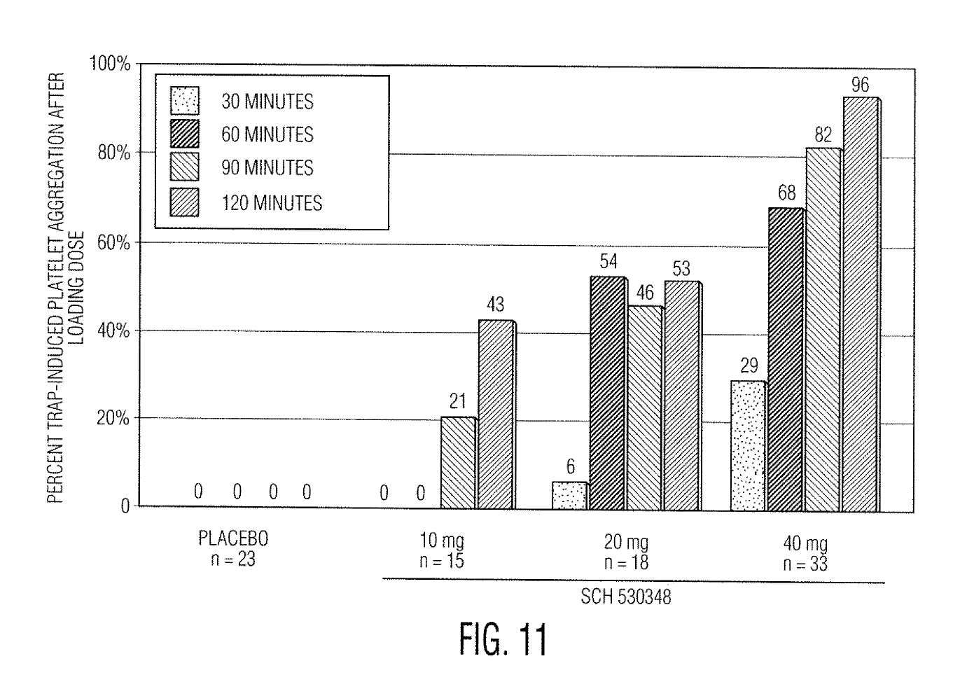

As demonstrated in FIGS. 7, 8 and 11, only the SCH 530348 40 mg loading dose afforded more than 90% of the tested population clinically relevant platelet aggregation inhibition within 2 hours. Approximately 68%, 82%, and 96% of patients had ≧80% inhibition of TRAP induced platelet aggregation at 1, 1.5, and 2 hours, respectively, using this loading dose. These results closely parallel the Phase 1 findings. In an ACS population or urgent PCI situation where early treatment and rapid inhibition is needed, the 40 mg dose appears to be the preferred loading dose since it achieves target platelet inhibition in such a large proportion of dosed patients.

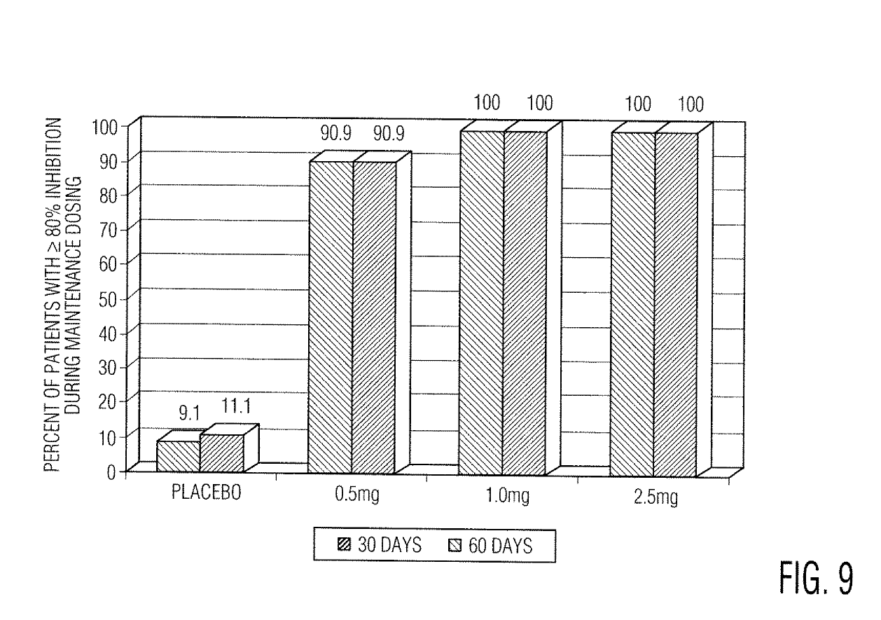

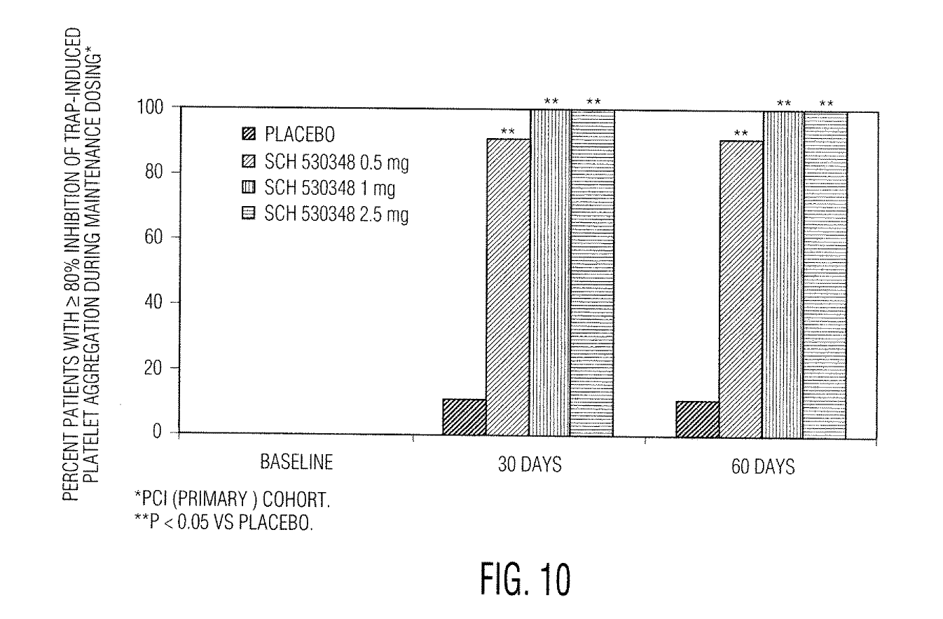

All three maintenance doses of SCH 530348 provided clinically relevant inhibition of platelet aggregation at thirty and sixty days, however, only the 1.0 and 2.5 mg/day regimens successfully achieved inhibition in 100% of the tested population (see FIGS. 9 and 10). When these results are coupled to the Phase 1 platelet aggregation studies, it would appear that the 2.5 mg daily regimen affords the broadest segment of the treated population clinically relevant platelet aggregation inhibition (as the maintenance regimen following a loading dose, and as the dose when urgent platelet aggregation inhibition is not clinically necessary) with a reasonable time to maximum effect. FIG. 10 demonstrates the sustained platelet aggregation effect of the maintenance dosing of SCH 530348.

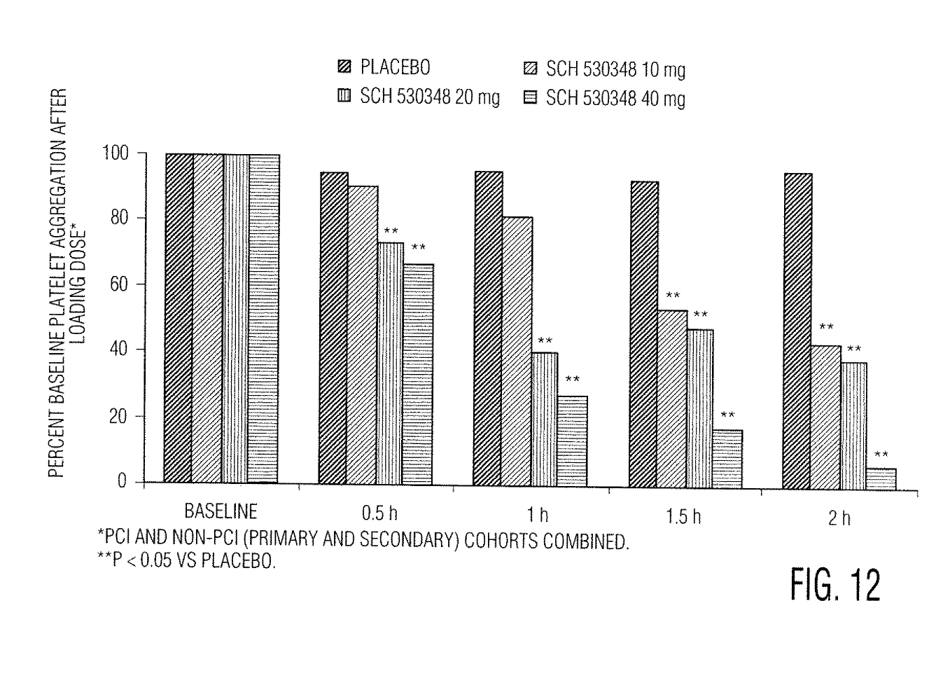

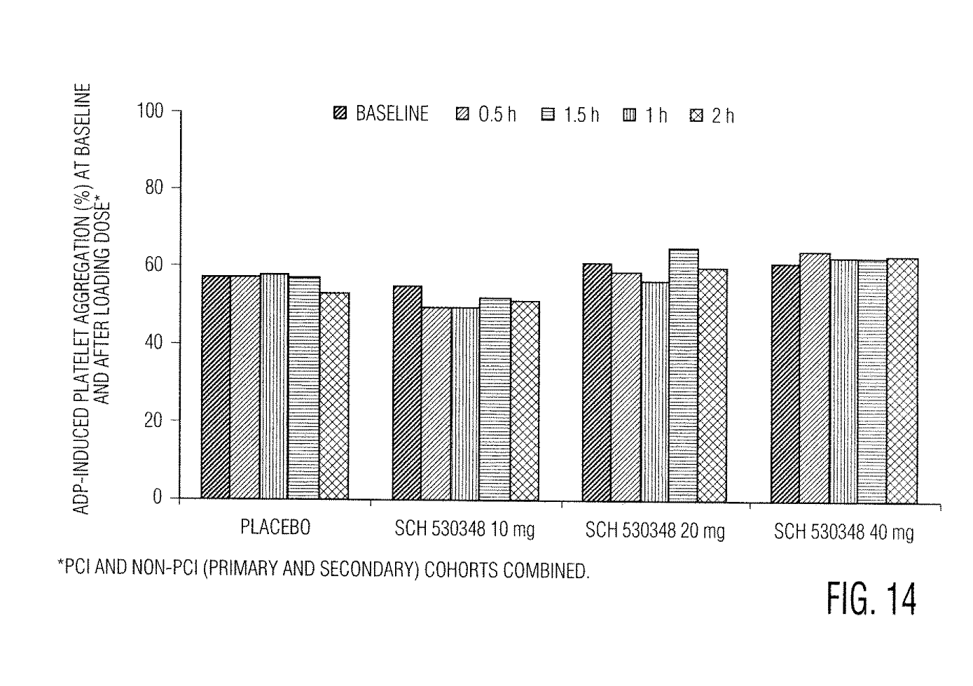

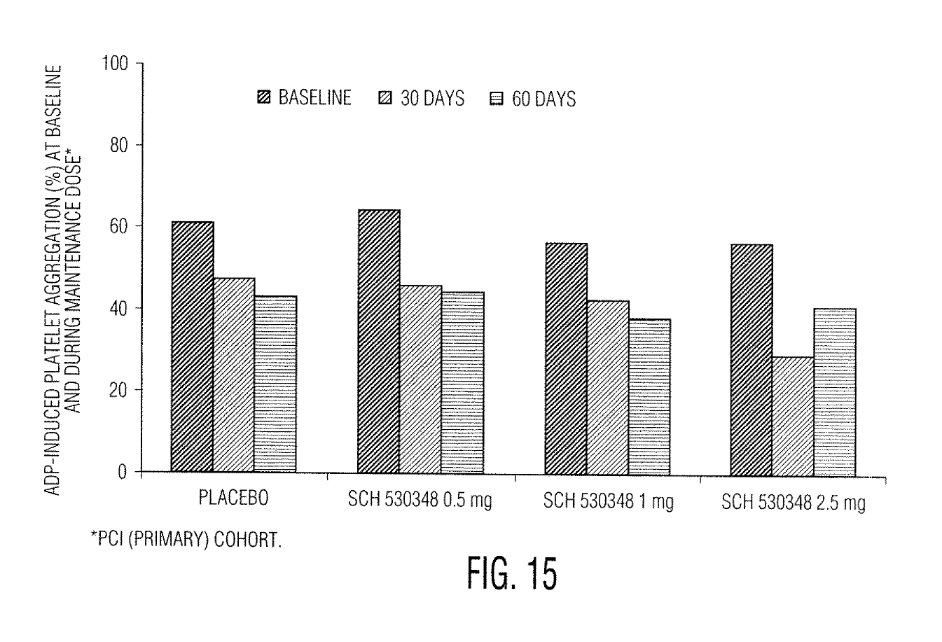

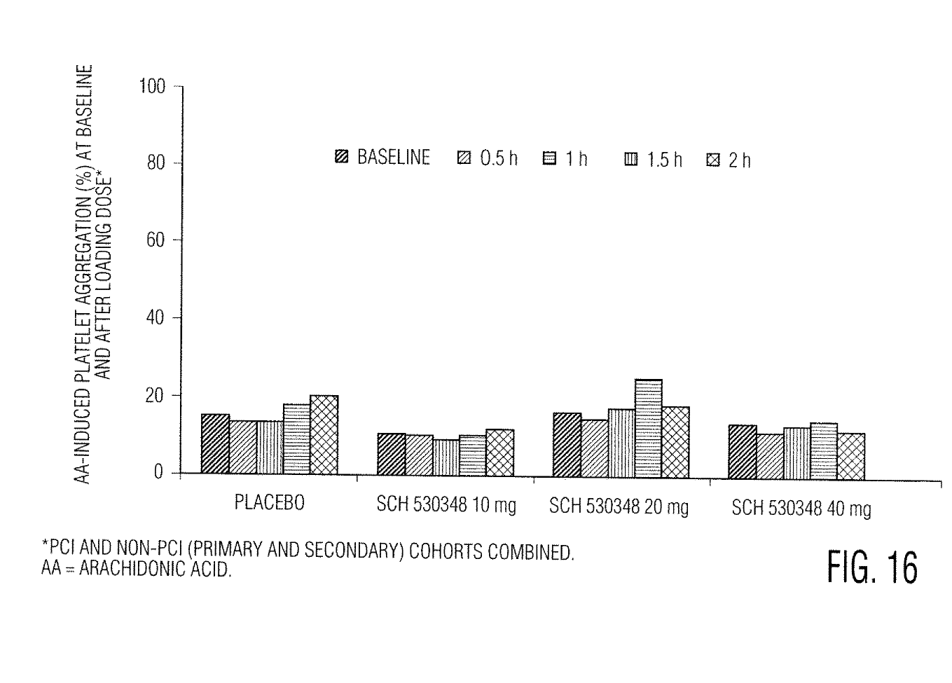

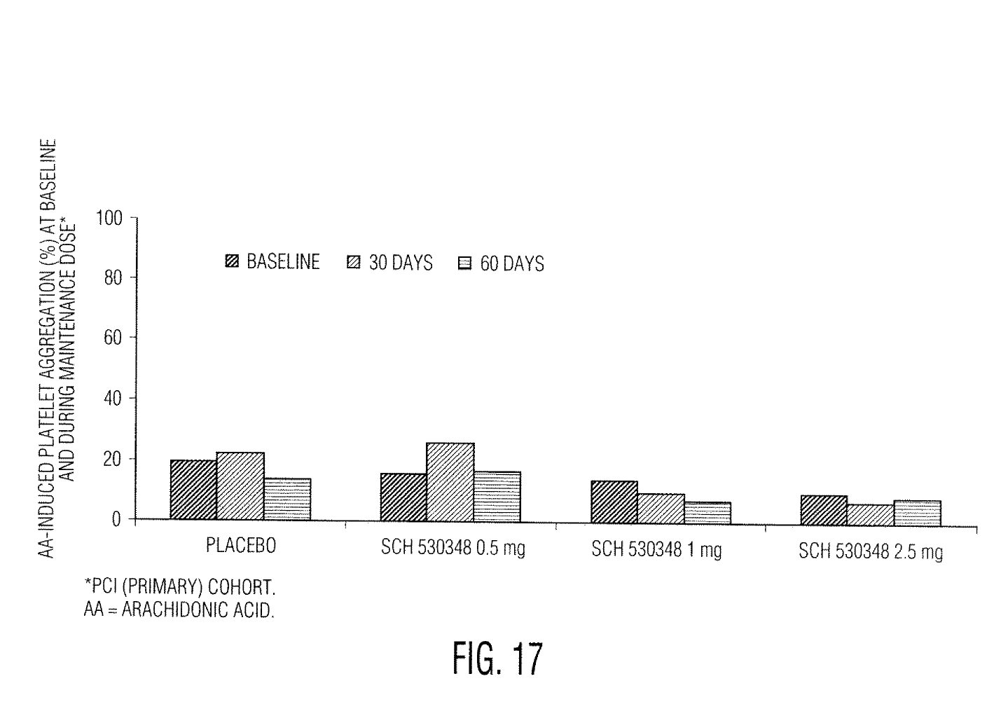

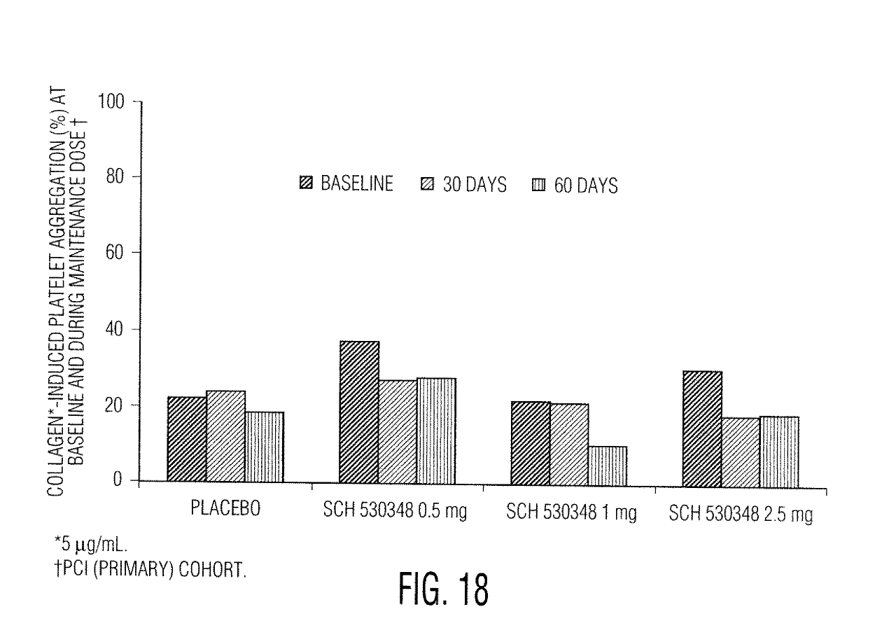

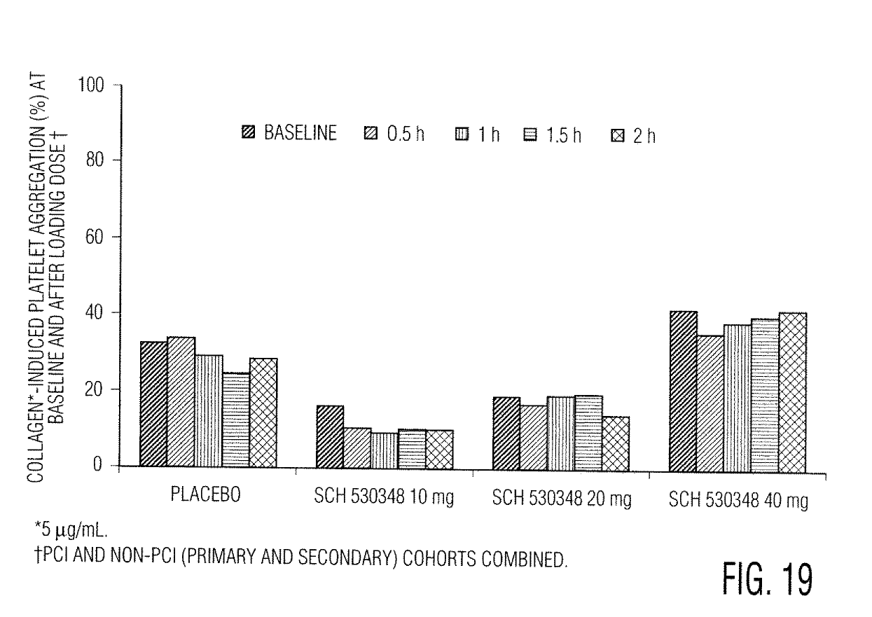

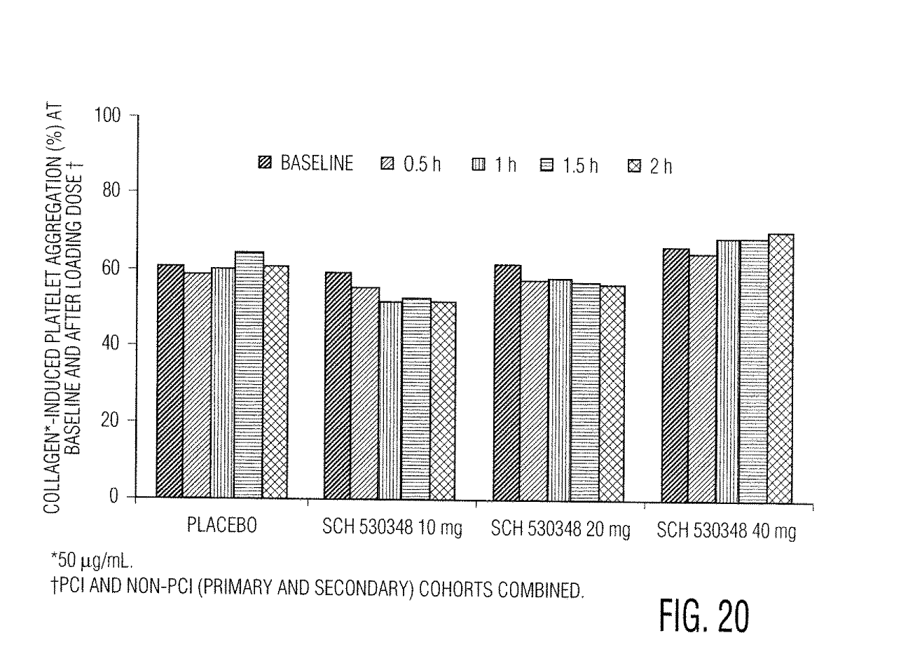

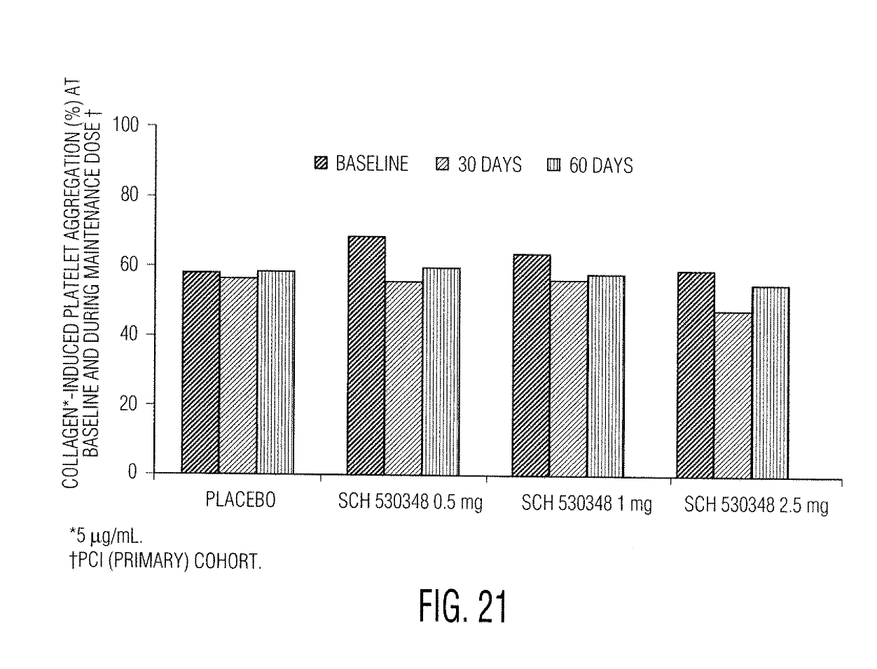

SCH 530348 exhibits dose-related inhibition of TRAP-induced platelet aggregation without activating platelets (i.e., without increasing expression of P-selectin or CD40 ligand), affecting coagulation parameters, or increasing bleeding time as measured by a modified Ivy method. This dose dependency is demonstrated in FIG. 12. Consistent with its mechanism of action, SCH 530348 has no effect on ADP-induced platelet aggregation, as demonstrated in FIGS. 14 and 15. Similarly, there is no effect on arachidonic acid (“AA”) -induced platelet aggregation (see FIGS. 16 and 17) or collagen-induced platelet aggregation (see FIGS. 18-21).

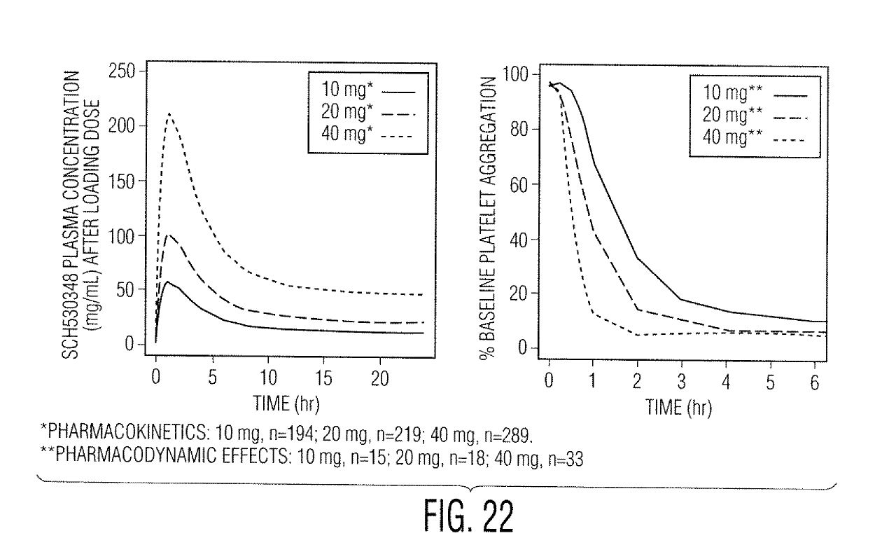

Single doses of SCH 530348 as low as 5 mg and multiple doses of 1 mg/day significantly inhibited TRAP-induced platelet aggregation. As the dose of SCH 530348 increased, the onset of platelet aggregation occurred at earlier sampling time points and maximum observed inhibition of aggregation increased, with 20 and 40 mg causing consistent maximum (>80%) inhibition of TRAP-induced platelet aggregation at 1-2 hours post dose. The pharmacokinetic and pharmacodynamic responses to the loading doses are demonstrated in FIG. 22. The duration of the inhibitory effect on platelets is dose- and concentration-dependent and is expected to last for at least two weeks after low single (3 and 5 mg) or multiple (1 and 2.5 mg) doses and persist for as long as eight weeks after single doses of 20 mg or 40 mg and multiple doses of ≧3 mg/day. The duration of these pharmacodynamic effects is consistent with the drug's long elimination half-life.

The pharmacokinetic and pharmacodynamic data displayed in FIG. 22 show that in as little as one hour after administration of the 40 mg loading dose, patients achieve peak blood concentration levels of SCH 530348 and therapeutically effective levels (at least 80%) of platelet inhibition. This level of platelet inhibition is believed to be sufficient to lower the risks of adverse clinical events associated with PCI. This rapid onset will allow “risk-abated PCI procedures” to be performed in as little as one hour after administration of the loading dose. The term “risk-abated PCI procedures” will be understood to mean those PCI procedures that are undertaken after the patient has achieved at least 80% platelet inhibition. This represents a marked improvement over the 4-6 hour period before risk-abated PCI can be performed using the current standard of care, i.e., clopidogrel and aspirin without a thrombin receptor antagonist.

Furthermore, clopidogrel is currently contra-indicated for cardiopulmonary bypass (“CPB”) procedures such as coronary arterial bypass graft (“CABG”) procedures due to bleed liability. Thus, if a patient is dosed with clopidogrel in anticipation of PCI, it is typical for any subsequently indicated CABG procedure (possibly in a time-critical circumstance) to be delayed for up to five days to allow the clopidogrel to clear from the patient's system. This five day delay may present a risk to the patient of suffering an intervening coronary event. Since the data presented herein support the view that SCH 530348 has limited bleed liability, a patient being treated with SCH 530348 alone for PCI would not be subject to such a delay in scheduling CPB, thus avoiding the risk of any intervening coronary event.

The main conclusions of the Phase 2 studies are as follows:
1. TIMI bleeding (major and minor) was not increased by SCH 530348 (3.3% placebo vs. 2.8% SCH 530348).
2. There was a dose-related (loading dose) numerical decrease in the composite of death and MACE (major adverse cardiac events) associated with SCH 530348 (8.6% placebo; SCH 530348 10 mg 8.5%, 20 mg 5%, 40 mg 4.6%), driven predominately by reductions in MI (7.3% placebo; SCH 530348 10 mg 5.4%, 20 mg 4.2%, 40 mg 3.5%).
3. There was a small, non-statically significant, increase in non-TIMI bleeding associated with SCH 530348 (32% placebo vs. 41% SCH 530348).
4. Increasing the loading dose of SCH 530348 led to earlier and more complete inhibition of TRAP-induced platelet aggregation within two hours of dose administration (96% of patients achieving >80% inhibition with 40 mg).

### Peripheral Percutaneous Interventional Procedures

Peripheral artery occlusive disease (“PAOD,” also known as peripheral vascular disease (“PVD”) and peripheral artery disease (“PAD”), is a collator for all diseases caused by the obstruction of large peripheral arteries, which can result from atherosclerosis, inflammatory processes leading to stenosis, an embolism or thrombus formation. It causes either acute or chronic ischemia. Common interventional treatments of PAD include the following:
Angioplasty (percutaneous transluminal angioplasty or “PTA”) can be done on solitary lesions in large arteries, such as the femoral artery. Peripheral angioplasty refers to the use of mechanical widening in opening blood vessels other than the coronary arteries. It is often called percutaneous transluminal angioplasty or PTA for short. PTA is most commonly done to treat narrowings in the leg arteries, especially the common iliac, external iliac, superficial femoral and popliteal arteries. PTA can also be done to treat narrowings in veins, etc.
Plaque excision, in which the plaque is scraped off of the inside of the vessel wall.
Occasionally, bypass grafting is needed to circumvent a seriously stenosed area of the arterial vasculature. Generally, the saphenous vein is used, although artificial material (e.g., Gore-Tex®) is often used for large tracts when the veins are of lesser quality.

These percutaneous interventional procedures (or peripheral percutaneous interventional procedures) used to treat PAD can be associated with adverse clinical events that are similar to those associated with PCI. Thus, the antithrombotic effects of thrombin receptor antagonists as described herein will have obvious utility in PTA, plaque excision and bypass grafting used to treat PAD.

### TRA Compounds

The present invention encompasses the use of any thrombin receptor antagonist to treat PCI patients. A variety of families of compounds have been shown to display activity as thrombin receptor antagonists. The compounds of Formula I have displayed such activity:

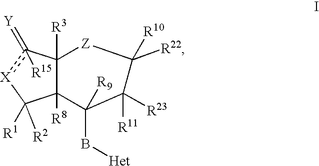

wherein the variables are as defined in U.S. Pat. No. 6,645,987, which is incorporated herein by reference.

As disclosed in U.S. publication no. 2004/0152736, a subset of particularly preferred compounds of Formula I is as follows:

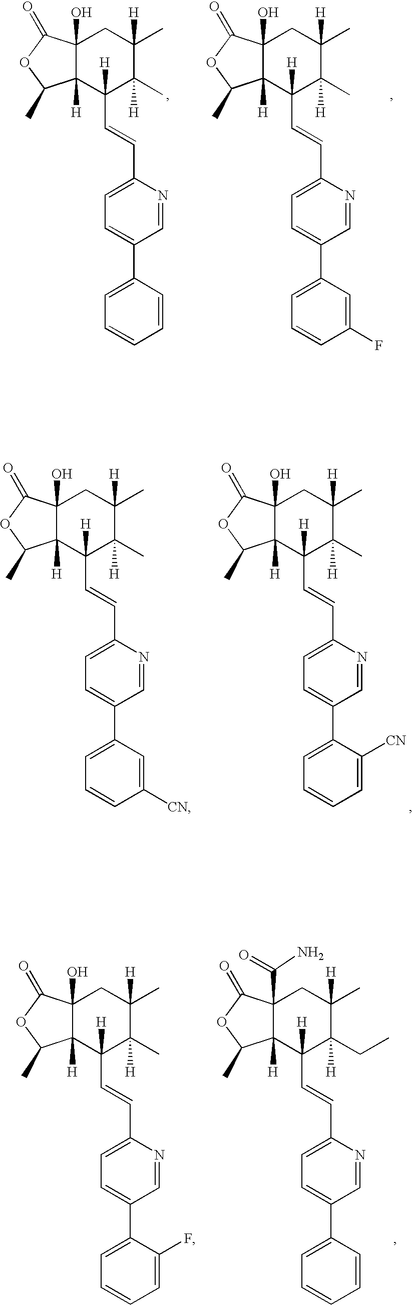

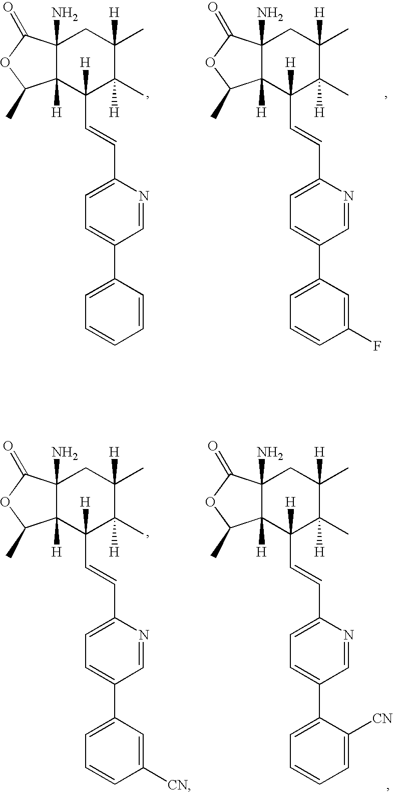

and the pharmaceutically acceptable isomers, salts, solvates and polymorphs thereof.

Further examples of active thrombin receptor antagonists are the compounds of Formula II, and pharmaceutically acceptable salts thereof:

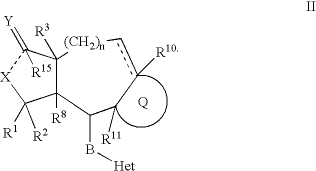

wherein the variables are as defined in U.S. Pat. No. 7,304,078, which is incorporated herein by reference. A particularly active and selective subset of thrombin receptor antagonists of Formula II is as follows:

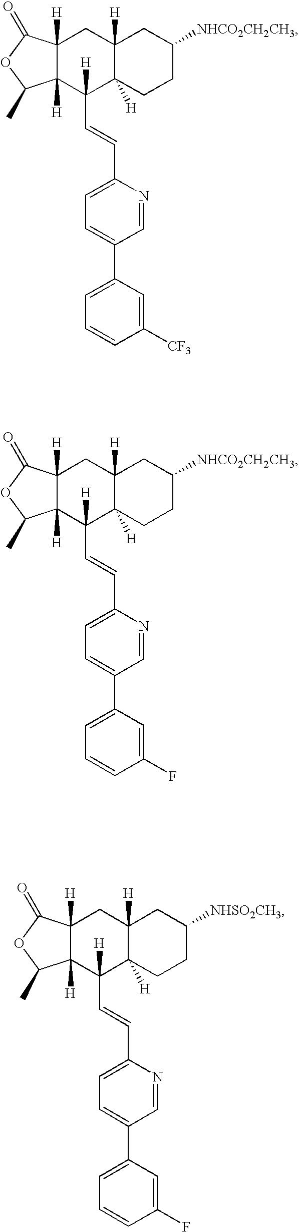

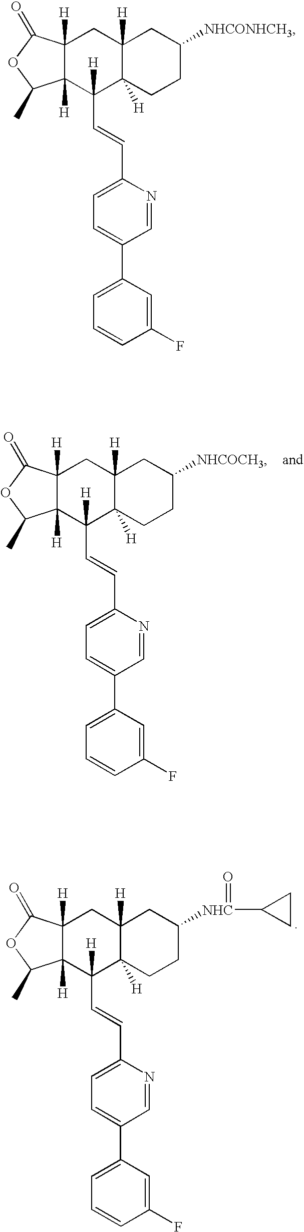

Among the more therapeutically promising thrombin receptor antagonist compounds of Formulas I and II are the following:

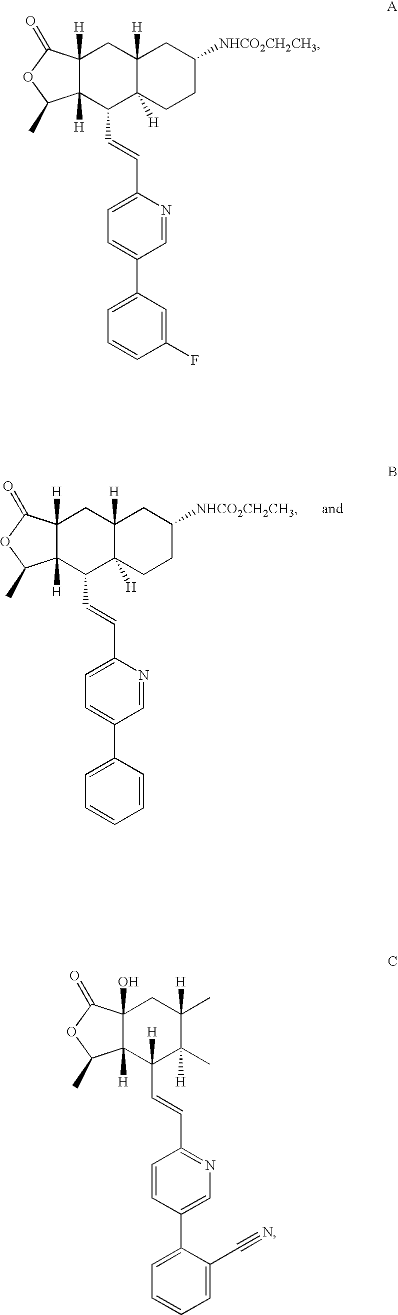

and the pharmaceutically acceptable isomers, salts, solvates and co-crystal forms thereof. Compound A is SCH 530348.

The bisulfate salt of SCH 530348 is currently in development as a thrombin receptor antagonist by Schering-Plough Corp. Its synthesis is disclosed in U.S. publication no. 03/0216437, which publication also discloses Compound C. Compound B is disclosed in U.S. Pat. No. 6,645,987.

Other TRA compounds for use in the methods of the present invention are disclosed in any of U.S. Pat. Nos. 6,063,847, 6,326,380, and 7,037,920, and U.S. Patent Publication nos. 20060079684 and 20060223808, the compound-related disclosures of which are all incorporated herein by reference in their entirety.

The above-described thrombin receptor antagonists are believed to exhibit excellent anti-platelet activity. In addition, they are believed to display a reduced bleeding liability relative to other platelet inhibiting agents, making them particularly attractive candidates as anti-platelet therapies in high bleeding risk scenarios. PCI presents precisely these requirements.

Any other agent that functions as a thrombin receptor antagonist is also within the scope of the present invention. For example, Eisai is currently developing an oral PAR-1 (protease activated receptor) antagonist, designated as E-5555, the structure of which is as follows:

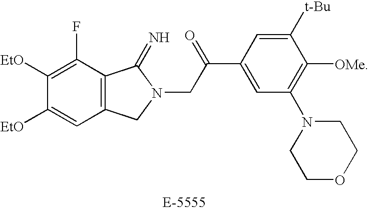

Cardiovascular agents that can be dosed in combination with TRA compounds in preventing adverse clinical events associated with percutaneous intervention include drugs that have anti-thrombotic, anti-platelet aggregation, antiatherosclerotic, antirestenotic and/or anti-coagulant activity. Such agents are useful in treating thrombosis-related diseases including thrombosis, atherosclerosis, restenosis, hypertension, angina pectoris, arrhythmia, heart failure, myocardial infarction, glomerulonephritis, thrombotic and thromboembolic stroke, peripheral vascular diseases, other cardiovascular diseases, cerebral ischemia, inflammatory disorders and cancer, as well as other disorders in which thrombin and its receptor play a pathological role. Suitable cardiovascular agents are selected from the group consisting of thromboxane A2 biosynthesis inhibitors such as non-stearoidal anti-inflammatories such as aspirin; thromboxane antagonists such as seratrodast, picotamide and ramatroban; adenosine diphosphate (ADP) inhibitors such as clopidogrel; cyclooxygenase inhibitors such as aspirin, meloxicam, rofecoxib and celecoxib; angiotensin antagonists such as valsartan, telmisartan, candesartran, irbesartran, losartan and eprosartan; endothelin antagonists such as tezosentan; phosphodiesterase inhibitors such as milrinoone and enoximone; angiotensin converting enzyme (ACE) inhibitors such as captopril, enalapril, enaliprilat, spirapril, quinapril, perindopril, ramipril, fosinopril, trandolapril, lisinopril, moexipril and benazapril; neutral endopeptidase inhibitors such as candoxatril and ecadotril; anticoagulants such as ximelagatran, fondaparin and enoxaparin; diuretics such as chlorothiazide, hydrochlorothiazide, ethacrynic acid, furosemide and amiloride; platelet aggregation inhibitors such as abciximab and eptifibatide; and GP IIb/IIIa antagonists.

Non-stearoidal anti-inflammatories include acetylsalicylic acid (Aspirin) amoxiprin, benorylate/benorilate, choline magnesium salicylate, diflunisal, ethenzamide, faislamine, methyl salicylate, magnesium salicylate, salicyl salicylate, alicylamide aceclofenac, acemetacin, alclofenac, bromfenac, etodolac, indometacin, nabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, phenylbutazone, sulfinpyrazone, piroxicam, droxicam, lornoxicam, meloxicam, tenoxicam, celecoxib, nimesulide, licofelone, and omega-3 fatty acids.

ADP inhibitors include any agents that act as inhibitors of adenosine diphosphate (“ADP”)-induced platelet aggregation, such as clopidogrel, marketed as PLAVIX®, ticlopidine, marketed as TICLID®, prasugrel, and AZD6140, which is in development for arterial thrombosis:

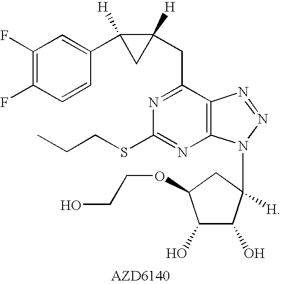

Preferred categories of cardiovascular agents for use in combination with TRA compounds include thromboxane A2 biosynthesis inhibitors, cyclooxygenase inhibitors and ADP antagonists. Especially preferred for use in the combinations are aspirin, clopidogrel, prasugrel and fragmin. Further TRA combination therapies are disclosed in U.S. publication no. 2001/0238674, which is herein incorporated in its entirety.

In these combinations of at least one thrombin receptor antagonist and one or more other therapeutically effective agent, the two or more active components may each be formulated individually and co-administered simultaneously or sequentially. The components of the combination can be administered individually or together in any conventional dosage form such as capsule, tablet, powder, cachet, suspension, solution, suppository, nasal spray, etc.

Alternatively, the active agents may be formulated in a single fixed-dose pharmaceutical composition comprising a thrombin receptor antagonist and the other therapeutically effective agent(s) along with a pharmaceutically acceptable carrier.

While the present invention has been described in conjunction with the specific embodiments set forth above, many alternatives, modifications, and variations thereof will be apparent to those of ordinary skill in the art. All such alternatives, modifications, and variations are intended to fall within the spirit and scope of the present invention.

We claim:

1. A method of preventing an adverse clinical event in a patient who is to undergo a percutaneous coronary intervention procedure comprising administering a therapeutically effective amount of a thrombin receptor antagonist to the patients

2. The method according to claim 1 wherein said adverse clinical event is a myocardial infarction, urgent revascularization, or ischemia requiring hospitalization.

3. The method according to claim 1 wherein said thrombin receptor antagonist is SCH 530348.

4. The method according to claim 3 wherein said therapeutically effective amount is administered as a loading dose of about 1 mg to about 5 mg.

5. The method according to claim 3 wherein said therapeutically effective amount is administered as a loading dose of about 2.5 mg.

6. The method according to claim 4 further comprising administering a maintenance dose of SCH 530348 to the patient once a day.

7. The method according to claim 6 wherein said maintenance dose is about 20 mg to about 40 mg.

8. The method according to claim 6 wherein said maintenance dose is about 40 my.

9. The method according to claim 1 wherein said thrombin receptor antagonist is the bisulfate salt of SCH 530348.

10. The method according to claim 17 wherein said thrombin receptor antagonist is selected from the group consisting of

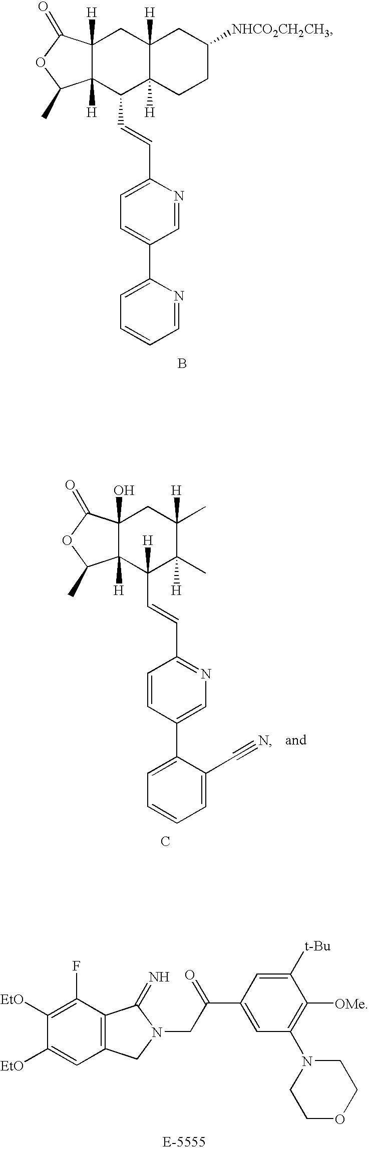

11. The method according to claim 1 further comprising administering to said patient an effective amount of a non-steroidal anti-inflammatory.

12. The method according to claim 11 wherein said non-steroidal anti-inflammatory is aspirin.

13. The method according to claim 1 further comprising administering to said patient an effective amount of an ADP antagonist.

14. The method according to claim 13 wherein said ADP antagonist is clopidogrel.

15. The method according to claim 13 wherein said ADP antagonist is prasugrel.

16. The method according to claim 1 wherein said thrombin receptor antagonist does not cause significant bleeding.

17. The method according to claim 16 wherein said bleeding is a TIMI major/minor bleed, a TIMI major bleed, or a TIMI minor bleed, or a combination thereof.

18. The method according to claim 1, wherein said percutaneous coronary intervention procedure is selected from the group consisting of balloon angioplasty, implantation of a stent, atherectomy, and brachytherapy.

19. The method according to claim 1, wherein said administration has no substantial effect on ADP-induced platelet aggregation.

20. The method according to claim 1, wherein said administration has no substantial effect on AA-induced platelet aggregation.

21. The method according to claim 1, wherein said administration has no substantial effect on collagen-induced platelet aggregation.

22. A method of preventing an adverse clinical event in a patient who is to undergo a percutaneous interventional procedure to treat peripheral artery disease comprising administering a therapeutically effective amount of a thrombin receptor antagonist to the patient.

23. The method according to claim 22 wherein said thrombin receptor antagonist is SCH 530348.

24. The method according to claim 23 wherein said therapeutically effective amount is administered as a loading dose of about 1 mg to about 5 mg.

25. The method according to claim 23 wherein said therapeutically effective amount is administered as a loading dose of about 2.5 mg.

26. The method according to claim 24 further comprising administering a maintenance dose of SCH 530348 to the patient once a day.

27. The method according to claim 26 wherein said maintenance dose is about 20 mg to about 40 mg.

28. The method according to claim 26 wherein said maintenance dose is about 40 mg.

29. The method according to claim 22 wherein said thrombin receptor antagonist is the bisulfate salt of SCH 530348.

30. The method according to claim 22, wherein said thrombin receptor antagonist is selected from the group consisting of 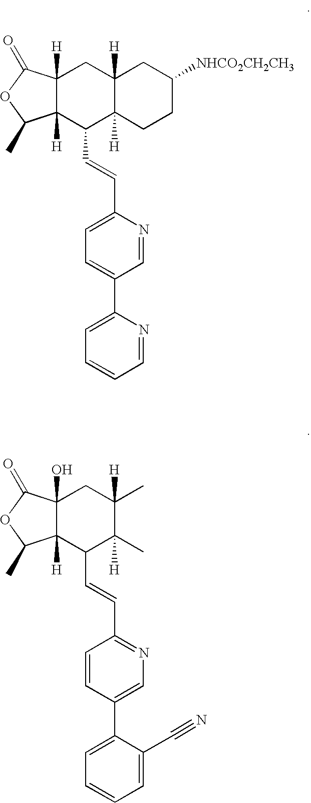
B, C, and

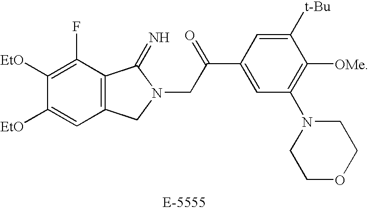

31. The method according to claim 22 further comprising administering to the patient an effective amount of a non-steroidal anti-inflammatory.

32. The method according to claim 31 wherein said non-steroidal anti-inflammatory is aspirin.

33. The method according to claim 22 further comprising administering to the patient an effective amount of an ADP antagonist.

34. The method according to claim 33 wherein said ADP antagonist is clopidogrel.

35. The method according to claim 33 wherein said ADP antagonist is prasugrel.

36. The method according to claim 22, wherein said percutaneous interventional procedure is selected from the group consisting of angioplasty, plaque excision and bypass grafting.

37. A method of achieving at least 80% platelet inhibition in a patient who is to undergo a percutaneous coronary interventional procedure or a peripheral percutaneous interventional procedure comprising administering to the patient a loading dose of about 40 mg of SCH 530348 at least one hour prior to the start of the procedure.
